(19) United States
(12) Patent Application Publication
(10) Pub. No.: US 2025/0226102 A1
(43) Pub. Date: Jul. 10, 2025

(54) TECHNIQUES FOR DETERMINING DOPAMINERGIC NEURAL CELL LOSS USING MACHINE LEARNING

(71) Applicant: Genentech, Inc., South San Francisco, CA (US)

(72) Inventors: Soumitra GHOSH, San Mateo, CA (US); Somaye Sadat Hashemifar, Sunnyvale, CA (US); Seyed Mohammadmohsen Hejrati, Stanford, CA (US)

(21) Appl. No.: 19/093,119

(22) Filed: Mar. 27, 2025

Related U.S. Application Data
(63) Continuation of application No. PCT/US2023/075162, filed on Sep. 26, 2023.
(60) Provisional application No. 63/500,562, filed on May 5, 2023.
(60) Provisional application No. 63/411,083, filed on Sep. 28, 2022.

(51) Int. Cl. G16H 50/20; A61B 5/00; G06T 7/00; G06T 7/11; G06T 7/174; G06T 7/194; G06T 11/00; G06V 10/24; G06V 10/32; G06V 10/40; G06V 10/77; G06V 10/774; G06V 10/82; G06V 20/70

(52) U.S. Cl. CPC G16H 50/20; G06T 7/0012; G06T 7/11; G06T 7/174; G06T 7/194; G06T 11/00; G06V 10/242; G06V 10/32; G06V 10/40; G06V 10/7715; G06V 10/774; G06V 20/70; A61B 5/4082; G06T 2207/10056; G06T 2207/10064; G06T 2207/20081; G06T 2207/20084; G06T 2207/30016; G06T 2207/30024; G06T 2207/30242; G06T 2210/41; G06V 10/82; G06V 2201/031

(57) ABSTRACT

Described herein are techniques for identifying regions of substantia nigra reticulata (SNR) and regions of substantia nigra compact dorsal (SNCD) in histology images and quantifying a number of dopaminergic neural cells within the images. In some embodiments, an image of a section of a brain may be input into a first machine learning model to obtain a first segmentation map comprising pixel-wise labels indicative of whether a corresponding pixel in the image depicts a region of SNR or SNCD. In some embodiments, the image (and, optionally, the first segmentation map) may also be input to a second machine learning model trained to generate a second segmentation map comprising pixel-wise labels indicating whether a corresponding pixel of the image depicts a dopaminergic neural cell or neural background tissue. The number of cells within the image may be determined based on the second segmentation map.

### CROSS-REFERENCE TO RELATED APPLICATIONS

This application is a continuation of International Application No. PCT/US2023/075162, filed on Sep. 26, 2023, which claims priority to U.S. Provisional Patent Application No. 63/411,083, entitled “Dopaminergic Neuron Analysis Using Deep Learning,” filed on Sep. 28, 2022, and U.S. Provisional Patent Application No. 63/500,562, entitled “Techniques for Determining Dopaminergic Neural Cell Loss Using Machine Learning,” filed on May 5, 2023, the disclosures of which are each incorporated herein by reference in their entireties.

### TECHNICAL FIELD

This application relates generally to determining dopaminergic neural cell loss using machine learning. In particular, this application includes techniques for identifying one or more regions of interests within a histology image depicting a section of a brain of a subject exhibiting dopaminergic neural cell loss. This application further includes techniques for segmenting and quantifying the dopaminergic neural cells within the histology image.

### BACKGROUND

Parkinson's disease (PD) is the second most common neurodegenerative disorder after Alzheimer's disease, affecting approximately 10 million people worldwide. The two hallmark signatures of PD are presence of Lewy bodies and the loss of dopaminergic neurons (DA). Patients with PD also can suffer from a plethora of motor neuron associated symptoms such as tremor, bradykinesia, rigid muscles, improper balance, automatic movements, loss of speech and writing ability, sleep disorders, loss of smell, and/or gastrointestinal problems. Both genetic and sporadic forms of PD depict a loss of dopaminergic neural cells. Within the brain, regions of Substantia Nigra (SN) and Ventral Tegmental Area (VTA) are known to harbor a majority of the dopaminergic neural cells. Loss of dopaminergic neural cells in regions of SN is considered a major trigger for development of PD symptoms. The regions of SN can be further sub-dissected into one or more regions of substantia nigra reticulata (SNR) and one or more regions of substantia nigra compacta dorsal (SNCD). The regions of SNR and SNCD correspond to the regions of the brain where dopaminergic neural cells, also referred to herein interchangeably as dopaminergic neurons, are most vulnerable. Currently, no therapy is available to halt or decrease the progression of PD.

Loss of dopaminergic neural cells is one of the major neuropathological end-points in drug efficacy preclinical PD studies. Analysis of dopaminergic neural cell loss in regions of SNR and SNCD requires careful annotations and drawing regions of interest (ROI) by a neuropathologist which further increases the duration of the study. In parallel, this also delays the process of making a go no-go decision for potential therapeutic targets. In the field of PD, the most advanced machine learning model can detect the nucleus of TH positive neurons in the entire brain 2D section but unable to segment the specific sub-region of the SN which are more susceptible to DA loss (e.g., the regions of SNR/SNCD). Thus, automated machine learning systems that can automatically identify regions of SNR and/or regions of SNCD within an image of the brain are needed.

Segmentation and quantification of dopaminergic neural cells within ROIs are crucial for experimental disease models and gene-function studies, particularly in PD-related studies. Traditionally, dopaminergic neural cells have been identified and counted manually by a trained pathologist. This process, however, is slow and can be biased due to the human element imparted by the trained pathologies. Therefore, the development of an unbiased, robust and faster turnaround pipeline is essential to the advancement of understanding PD progression in a subject.

The success of deep learning models in image segmentation naturally suggests that segmentation models be developed for dopaminergic neural cell segmentation in medical images. The developed models can be further optimized to separate adjacent dopaminergic neural cells for automatic quantification thereof. However, challenges exist in developing such models due to the training data being noisy and small, the preprocessing of the images, and variability in dopaminergic neural cell morphology.

Preclinical research has illustrated that PD is highly dependent on segmentation and quantification of dopaminergic neural cells within one or more ROIs of the brain (e.g., regions of SNR/SNCD). These regions are known to be highly sensitive to genetic alterations. Analyzing and quantifying dopaminergic neural cells in these regions is necessary to understand animal models of PD and to determine the efficacy of PD-aimed therapeutics. Thus, automated machine learning systems for the segmentation and quantification of dopaminergic neural cells in regions of SNR and/or SNCD of a subject having PD are needed.

### SUMMARY

Described herein are techniques for identifying regions of SNR and regions of SNCD in images of a subject with dopaminergic neural cell loss. Subjects diagnosed with PD tend to have higher dopaminergic neural cell loss than subjects who have not been diagnosed with PD. Dopaminergic neural cell loss can present as a loss of TH signal. The techniques enable the regions of SNR and/or SNCD to be identified independent of TH signal. Also described herein are techniques for segmenting and quantifying dopaminergic neural cells within one or more ROIs of the brain, such as regions of SNR and SNCD. As an example, subjects diagnosed with PD tend to have higher dopaminergic neural cell loss than patients who have not been diagnosed with PD. Thus, a health state of a subject can be estimated based on the quantification of the dopaminergic neural cells within the ROIs.

In some embodiments, methods for identifying regions of SNR and regions of SNCD in images of a subject (a preclinical PD mouse model) with dopaminergic neural cell loss are described. For example, subjects diagnosed with PD commonly experience dopaminergic neural cell loss. The methods may include, in one or more examples, receiving an image depicting a section of a brain including substantia nigra (SN) of the subject. A segmentation map of the image may be obtained by inputting the image into a trained machine learning model. The segmentation map may comprise a plurality of pixel-wise labels. Each pixel-wise label may be indicative of a portion of one or more regions of SNR, a portion of one or more regions of SNCD, or non-SN brain tissue. In one or more examples, one or more regions of SNR and one or more regions of SNCD may be identified based on the segmentation map of the image.

In some embodiments, methods for determining a number of dopaminergic neural cells within images depicting a section of a brain of a subject with dopaminergic neural cell loss are described. For example, subjects diagnosed with PD commonly experience dopaminergic neural cell loss. The methods may include, in one or more examples, receiving an image depicting a section of the brain and dividing the image into a plurality of patches. Using a trained machine learning model, a segmentation map for each patch of the plurality of patches may be generated. In one or more examples, the segmentation map may comprise a plurality of pixel-wise labels. Each pixel-wise label may be indicative of whether a corresponding pixel in the image is classified as depicting dopaminergic neural cells or neural background tissue. In one or more examples, the number of dopaminergic neural cells within the image may be identified based on the segmentation map generated for each of the plurality of patches.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed can be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an example system for identifying regions of SNR and SNCD within an image, and segmenting and quantifying dopaminergic neural cells within those regions, in accordance with various embodiments.

FIG. 2 illustrates an example of an SN segmentation model used to generate a segmentation map indicating regions of SNR and SNCD within an image, in accordance with various embodiments.

FIG. 3 illustrates an example training process for training an SN segmentation model, in accordance with various embodiments.

FIG. 4 illustrates an example of a dopaminergic neural cell segmentation and quantification model used to generate a segmentation map indicating detected dopaminergic neural cells and a number of dopaminergic neural cells detected, in accordance with various embodiments.

FIG. 5 illustrates an example of the training process for training a dopaminergic neural cell segmentation and quantification model, in accordance with various embodiments.

FIG. 6 illustrates an example architecture of the dopaminergic neural cell segmentation and quantification model of FIG. 4, in accordance with various embodiments.

FIG. 7 illustrates an example machine learning pipeline for identifying regions of SNR and SNCD within an image, and segmenting and quantifying dopaminergic neural cells within those regions, in accordance with various embodiments.

FIG. 8 illustrates a flowchart of an example method for identifying regions of SNR and regions of SNCD within an image, in accordance with various embodiments.

FIG. 9 illustrates a flowchart of an example method for determining a number of dopaminergic neural cells within an image, in accordance with various embodiments.

FIG. 10 illustrates an example image of a section of a brain of a subject, a ground truth mask indicating an SNR region for the image, and a model-predicted mask of the SNR region for the image, in accordance with various embodiments.

FIG. 11 illustrates an example image of a section of a brain of a subject, a ground truth mask indicating an SNCD region for the image, and a model-predicted mask of the SNCD region for the image, in accordance with various embodiments.

FIGS. 12A-12B illustrate example images of a section of a brain of a subject, a ground truth mask indicating an SNR and an SNCD region for the image, and a model-predicted mask of the SNR and the SNCD region for the image, in accordance with various embodiments.

FIGS. 13A-13B illustrate an example image of a section of a brain of a subject and a zoomed-in portion of the image including annotations of an SNR and an SNCD region of the brain, respectively, in accordance with various embodiments.

FIG. 14 illustrates example images of a region of interest of a section of a brain of a subject, a ground truth mask of dopaminergic neural cells based on the image, and a model-predicted mask of dopaminergic neural cells based on the image, in accordance with various embodiments.

FIG. 15 illustrates example images of dopaminergic neural cells including ground truth indications of the dopaminergic neural cells, correctly predicted indications of the dopaminergic neural cells, and incorrectly predicted indications of the dopaminergic neural cells, in accordance with various embodiments.

FIG. 16 illustrates an example image depicting a section of a brain of subject including model-predicted and ground-truth indications of dopaminergic neural cells, in accordance with various embodiments.

FIG. 17 illustrates a zoomed-in portion of an image of a section of a brain of subject including annotations indicating clusters of dopaminergic neural cells, in accordance with various embodiments.

FIG. 18 illustrates an example computer system used to implement some or all of the techniques described herein.

### DETAILED DESCRIPTION

Described herein are systems, methods, and programming describing a machine learning pipeline for identifying regions of substantia nigra reticulata (SNR) and regions of substantia nigra compacta dorsal (SNCD) in images of a subject, segmenting dopaminergic neural cells within these images, and quantifying a number of dopaminergic neural cells within these images. In some embodiments, the subject may have dopaminergic neural cell loss within regions of substantia nigra (SN). For example, patients diagnosed with Parkinson's disease (PD) commonly have a loss of dopaminergic neural cells within regions of SN. The images may be histology images, which can also be referred to as digital pathology images. Accordingly, as used herein, the term “image” or “images” includes histology images and digital pathology images (unless otherwise indicated (e.g., non-medical images)).

Parkinson's disease (PD) is a neurodegenerative disorder affecting approximately 10 million people worldwide. One of the hallmarks of PD is the loss of dopaminergic neural cells. Both genetic and sporadic forms of PD depict a loss of dopaminergic neural cells. Within the brain, regions of substantia nigra (SN) and ventral tegmental area (VTA) are known to harbor a majority of the dopaminergic neural cells. Loss of dopaminergic neural cells in regions of SN is considered a major trigger for development of PD symptoms. The regions of SN can be dissected into regions of SNR, regions of SNCD, and/or regions of non-SN brain tissue.

Analysis of dopaminergic neural cell loss in the regions of SNR and SNCD requires careful annotations and drawing regions of interest (ROI) by a trained neuropathologist. This is a time consuming process that forms a significant bottleneck in PD research. Additionally, trained neuropathologists may introduce bias into the analysis. For example, a first pathologist may annotate an image of a section of a brain to outline a region of SNR while a second pathologist may annotate the image with a different outline of the region of SNR.

In the field of PD, existing machine learning models can detect the nucleus of TH positive neurons (e.g., dopaminergic neural cells) in the images of the brain, however these models are unable to segment the specific sub-region of the SN which are more susceptible to DA loss (e.g., the regions of SNR/SNCD). Thus, automated machine learning systems that can automatically identify regions of SNR and/or regions of SNCD within an image of the brain are needed.

Segmentation and quantification of dopaminergic neural cells within ROIs are crucial for experimental disease models and gene-function studies, particularly in PD-related studies. Traditionally, dopaminergic neural cells have been identified and counted manually by a trained pathologist. This process, however, is slow and, similar to the SNR/SNCD segmentation task, can be biased when performed by trained pathologies. Therefore, the development of an unbiased, robust, and faster turnaround pipeline is essential to the advancement of understanding PD progression in a subject.

The success of deep learning models in image segmentation naturally suggests that segmentation models be developed for dopaminergic neural cell segmentation in medical images. The developed models can be further optimized to separate adjacent dopaminergic neural cells for automatic quantification thereof. However, challenges exist in developing such models due to the training data being noisy and small, the preprocessing of the images, and variability in dopaminergic neural cell morphology.

Preclinical research into PD is highly dependent on segmentation and quantification of dopaminergic neural cells within one or more ROIs of the brain (e.g., regions of SNR/SNCD). These regions are known to be highly sensitive to genetic alterations. Analyzing and quantifying dopaminergic neural cells in these regions is necessary to understand animal models of PD and to determine the efficacy of PD-aimed therapeutics. Thus, automated machine learning systems for the segmentation and quantification of dopaminergic neural cells in regions of SNR and/or SNCD of a subject having PD are needed.

As described herein, the term “subject” refers to an animal model, such as, for example, mice or other preclinical animal models. Some embodiments comprise a “subject” being other animals such as, for example, rats, monkeys, or humans.

In some embodiments, an exemplary system can train one or more models using histology images depicting dopaminergic neurons in various preclinical models (e.g., rats, monkeys, and/or humans). Accordingly, the models can be used quantify dopaminergic neural cells loss for the various preclinical models (e.g., rats, monkeys, and/or humans).

### Technical Advantages

In the field of PD, it is known that the loss of dopaminergic neural cells in regions of SNR and SNCD is a major neuropathological end-point for drug efficacy in preclinical studies. However, the analysis of the regions of SNR and SNCD requires careful annotations and drawing regions of interests (ROIs) by highly-trained neuropathologists. This results in a significant bottleneck when decisions need to be made regarding potential therapeutic targets. Currently, no known machine learning models exist that allow for a fast, unbiased analysis of digital pathology images depicting SN to segment regions of SNR/SNCD within the images and annotate those images to indicate the locations of the regions of SNR and SNCD.

Embodiments described herein may be configured to identify regions of substantia nigra reticulata (SNR) and regions of substantia nigra compacta dorsal (SNCD) in images of a subject diagnosed with Parkinson's disease (PD). In particular, images depicting a section of a brain including SN of a subject may be received. The image may be fed to a trained machine learning model to obtain a segmentation map of the image, where the segmentation may comprise a plurality of pixel-wise labels each being indicative of a portion of a region of one or more regions of SNR, a portion of one or more regions of SNCD, or non-SN brain tissue. One or more regions of SNR and one or more regions of SNCD may be identified based on the segmentation map of the image.

Accordingly, some embodiments described herein provide technical advantages over existing techniques for analyzing digital pathology images to identify regions of SNR/SNCD with minimal latency. The quantitative and qualitative results described herein how the disclosed embodiments can be implemented to replace laborious time consuming expert labeling of pathology images to advance preclinical research. Additionally, the embodiments described herein can solve one of the major problems in medical imaging that arises from pathologist-based associated bias. Using highly accurate machine learning model(s), as described herein, can deliver unbiased data in a short time to segment anatomical sub-regions for 2D images (e.g., regions of SNR/SNCD), thereby eliminating pathologist-induced bias from one study to another. Another advantage of the described embodiments include detecting the regions of SNR and SNCD independent of TH signal level. This enables ROIs to be detected within images of the brain sections independent of the TH signal. For example, for a brain tissue stained for another end-point pathological marker or biomarker, the expression of that marker specifically in the SN with this pipeline can be evaluated.

It is also known that, particularly within the field of PD, segmenting and quantifying dopaminergic neural cells within regions of interest, such as regions of SNR/SNCD, are crucial for experimental disease models and gene-function studies. Traditionally, neural cells have been drawn and counted manually by expertly-trained pathologists. However, similar to the issues mentioned with respect to SNR/SNCD identification, this produces a large bottleneck in the analysis pipeline, leading to delays when determining drug efficacy and for drug discovery. Additionally, trained pathologists can, even unknowingly, introduce bias into the results.

Embodiments described herein may be configured to determine a number of dopaminergic neural cells within an image of a section of a brain of a subject diagnosed with PD. In particular, an image depicting a section of the brain may be received and divided into a plurality of patches. Using a trained machine learning model, a segmentation map may be generated for each of the plurality of patches. The segmentation map may include a plurality of pixel-wise labels each being indicative of whether a corresponding pixel from the image is classified as depicting a dopaminergic neural cell or neural background tissue. The number of dopaminergic neural cells within the image may be determined based on the segmentation map generated for each of the patches.

Accordingly, some embodiments described herein provide technical advantages over existing techniques for analyzing digital pathology images to identify and quantify dopaminergic neural cells. In particular, the identification and quantification techniques may be trained to focus on one or more ROIs within the image, such as regions of SNR/SNCD.

An additional technique advantage provided by the disclosed embodiments is the ability to use non-medical and medical images to train the various machine learning models. Annotated digital pathology images indicating regions of SNR/SNCD and/or dopaminergic neural cells are limited. Some embodiments described herein are capable of performing initial machine learning training using non-medical images followed by a self-supervised learning and transfer learning step to fine-tune the model using medical images.

### Example System

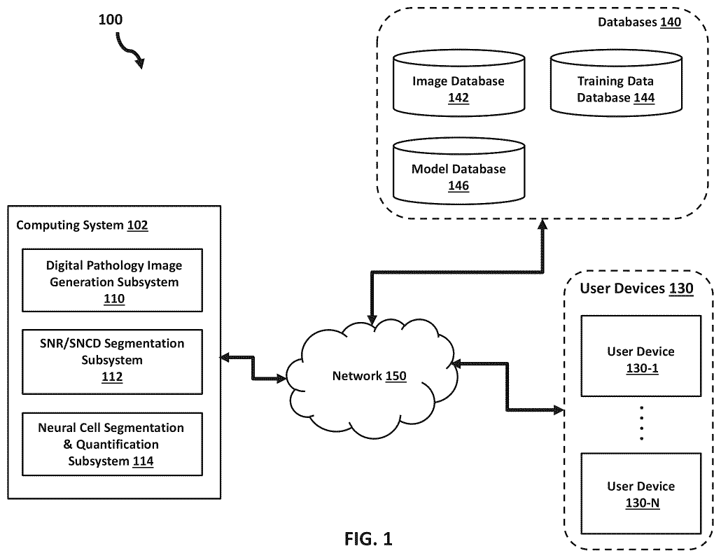

FIG. 1 illustrates an example system for identifying regions of SNR and SNCD within an image, and segmenting and quantifying dopaminergic neural cells within those regions, in accordance with various embodiments. System 100 may include a computing system 102, user devices 130-1 to 130-N (also referred to collectively as “user devices 130” and individually as “user device 130”), databases 140 (e.g., image database 142, training data database 144, model database 146), or other components. In some embodiments, components of system 100 may communicate with one another using network 150, such as the Internet.

User devices 130 may communicate with one or more components of system 100 via network 150 and/or via a direct connection. User devices 130 may be a computing device configured to interface with various components of system 100 to control one or more tasks, cause one or more actions to be performed, or effectuate other operations. For example, user device 130 may be configured to receive and display an image of a scanned biological sample. Example computing devices that user devices 130 may correspond to include, but are not limited to, which is not to imply that other listings are limiting, desktop computers, servers, mobile computers, smart devices, wearable devices, cloud computing platforms, or other client devices. In some embodiments, each user device 130 may include one or more processors, memory, communications components, display components, audio capture/output devices, image capture components, or other components, or combinations thereof. Each user device 130 may include any type of wearable device, mobile terminal, fixed terminal, or other device.

It should be noted that while one or more operations are described herein as being performed by particular components of computing system 102, those operations may, in some embodiments, be performed by other components of computing system 102 or other components of system 100. As an example, while one or more operations are described herein as being performed by components of computing system 102, those operations may, in some embodiments, be performed by aspects of user devices 130. It should also be noted that, although some embodiments are described herein with respect to machine learning models, other prediction models (e.g., statistical models or other analytics models) may be used in lieu of or in addition to machine learning models (e.g., a statistical model replacing a machine-learning model and a non-statistical model replacing a non-machine-learning model in one or more embodiments). Still further, although a single instance of computing system 102 is depicted within system 100, additional instances of computing system 102 may be included (e.g., computing system 102 may comprise a distributed computing system).

Computing system 102 may include a digital pathology image generation subsystem 110, an SNR/SNCD segmentation subsystem 112, a neural cell segmentation and quantification subsystem 114, or other components. Each of digital pathology image generation subsystem 110, SNR/SNCD segmentation subsystem 112, and neural cell segmentation and quantification subsystem 114 may be configured to communicate with one another, one or more other devices, systems, and/or servers, using network 150 (e.g., the Internet, an Intranet). System 100 may also include one or more databases 140 (e.g., image database 142, training data database 144, model database 146) used to store data for training machine learning models, storing machine learning models, or storing other data used by one or more components of system 100. This disclosure anticipates the use of one or more of each type of system and component thereof without necessarily deviating from the teachings of this disclosure.

Although not illustrated, other intermediary devices (e.g., data stores of a server connected to computing system 102) can also be used. The components of system 100 of FIG. 1 can be used in a variety of contexts where scanning and evaluating digital pathology images, such as whole slide images, are essential components of the work. As an example, system 100 can be associated with a clinical environment where a user is evaluating the sample for possible diagnostic purposes. The user can review the image using user device 130 prior to providing the image to computing system 102. The user can provide additional information to computing system 102 that can be used to guide or direct the analysis of the image. For example, the user can provide a prospective diagnosis or preliminary assessment of features within the scan. The user can also provide additional context, such as the type of tissue being reviewed. As another example, system 100 can be associated with a laboratory environment where tissues are being examined, for example, to determine the efficacy or potential side effects of a drug. In this context, it can be commonplace for multiple types of tissues to be submitted for review to determine the effects on the whole body of said drug. This can present a particular challenge to human scan reviewers, who may need to determine the various contexts of the images, which can be highly dependent on the type of tissue being imaged. These contexts can optionally be provided to computing system 102.

In some embodiments, digital pathology image generation subsystem 110 may be configured to generate one or more whole slide images or other related digital pathology images, corresponding to a particular sample. For example, an image generated by digital pathology image generation subsystem 110 may include a stained section of a biopsy sample. As another example, an image generated by digital pathology image generation subsystem 110 may include a slide image (e.g., a blood film) of a liquid sample. As yet another example, an image generated by digital pathology image generation subsystem 110 can include fluorescence microscopy such as a slide image depicting fluorescence in situ hybridization (FISH) after a fluorescent probe has been bound to a target DNA or RNA sequence. Digital pathology image generation subsystem 110 may include one or more systems, modules, devices, or other components.

Digital pathology image generation subsystem 110 may be configured to prepare a biological sample for digital pathology analyses. Some example types of samples include biopsies, solid samples, samples including tissue, or other biological samples. Biological samples may be obtained for subjects with PD. For example, the subjects may be participating in one or more clinical trials.

Digital pathology image generation subsystem 110 may be configured to fix and/or embed a sample. In some embodiments, digital pathology image generation subsystem 110 may facilitate infiltrating a sample with a fixating agent (e.g., liquid fixing agent, such as a formaldehyde solution) and/or embedding substance (e.g., a histological wax). Digital pathology image generation subsystem 110 may include one or more systems, subsystems, modules, or other components, such as a sample fixation system, a dehydration system, a sample embedding system, or other subsystems. In one or more examples, the sample fixation system may be configured to fix a biological sample. Fixing the sample may include exposing the sample to a fixating agent for at least a threshold amount of time (e.g., at least 3 hours, at least 6 hours, at least 13 hours, etc.). In one or more examples, the dehydration system may be configured to dehydrate the biological sample. For example, dehydrating the sample may include exposing the fixed sample and/or a portion of the fixed sample to one or more ethanol solutions. In some embodiments, the dehydration system may also be configured to clear the dehydrated sample using a clearing intermediate agent. An example clearing intermediate agent may include ethanol and a histological wax. In one or more examples, the sample embedding system may be configured to infiltrate the biological sample. The sample may be infiltrated using a heated histological wave (e.g., liquid). In some embodiments, the sample embedding system may perform the infiltration process one or more times for corresponding predefined time periods. The histological wax can include a paraffin wax and potentially one or more resins (e.g., styrene or polyethylene). Digital pathology image generation subsystem 110 may further be configured to cool the biological sample and wax or otherwise allow the biological sample and wax to be cooled. After cooling, the wax-infiltrated biological sample may be blocked out.

In some embodiments, digital pathology image generation subsystem 110 may be configured to receive the fixed and embedded sample and produce a set of sections. The fixed and embedded sample may be exposed to cool or cold temperatures. In one or more examples, digital pathology image generation subsystem 110 may include a sample slicer configured to cut the chilled sample (or a trimmed version thereof) to produce a set of sections. For example, each section may have a thickness that is less than 100 μm, less than 50 μm, less than 10 μm, less than 5 μm, or other dimensions. As another example, each section may have a thickness that is greater than 0.1 μm, greater than 1 μm, greater than 2 μm, greater than 4 μm, or other dimensions. The sections may have the same or similar thickness as the other sections. For example, a thickness of each section may be within a threshold tolerance (e.g., less than 1 μm, less than 0.1 μm, less than 0.01 μm, or other values). The cutting of the chilled sample can be performed in a warm water bath (e.g., at a temperature of at least 30° C., at least 35° C., at least 40° C., or other temperatures).

Digital pathology image generation subsystem 110 may be configured to stain one or more of the sample sections. The staining may expose each section to one or more staining agents. Example staining agents include background nucleus stains, such as Nissl (which stains light blue) and Thionine (which stains violet). Another example staining agent includes tyrosine hydroxylase (TH) enzyme, which acts as an indicator of dopaminergic neuron viability.

In some embodiments, digital pathology image generation subsystem 110 may include an image scanner. Each of the stained sections can be presented to the image scanner, which can capture a digital image of that section. In one or more examples, the image scanner may include a microscope camera. The image scanner may be configured to capture a digital image at one or more levels of magnification (e.g., 5× magnification). Manipulation of the image can be used to capture a selected portion of the sample at the desired range of magnifications. In some embodiments, annotations to exclude areas of assay, scanning artifacts, and/or large areas of necrosis may be performed (manually and/or with the assistance of machine learning models). Digital pathology image generation subsystem 110 can further capture annotations and/or morphometrics identified by a human operator. In some embodiments, a section may be returned after one or more images are captured such that the section can be washed, exposed to one or more other stains, and imaged again.

It will be appreciated that one or more components of digital pathology image generation subsystem 110 can, in some instances, operate in connection with human operators. For example, human operators can move the sample across various components of digital pathology image generation subsystem 110 and/or initiate or terminate operations of one or more subsystems, systems, or components of digital pathology image generation subsystem 110. As another example, part or all of one or more components of the digital pathology image generation system can be partly or entirely replaced with actions of a human operator.

Further, it will be appreciated that, while various described and depicted functions and components of digital pathology image generation subsystem 110 pertain to processing of a solid and/or biopsy sample, other embodiments can relate to a liquid sample (e.g., a blood sample). For example, digital pathology image generation subsystem 110 can receive a liquid-sample (e.g., blood or urine) slide that includes a base slide, smeared liquid sample, and a cover. In some embodiments, digital pathology image generation subsystem 110 may include an image scanner to capture an image (or instruct an image scanner to capture the image) of the sample slide. Furthermore, some embodiments of digital pathology image generation subsystem 110 include capturing images of samples using advancing imaging techniques. For example, after a fluorescent probe has been introduced to a sample and allowed to bind to a target sequence, appropriate imaging techniques can be used to capture images of the sample for further analysis.

A given sample can be associated with one or more users (e.g., one or more physicians, laboratory technicians and/or medical providers) during processing and imaging. An associated user can include, by way of example and not of limitation, a person who ordered a test or biopsy that produced a sample being imaged, a person with permission to receive results of a test or biopsy, or a person who conducted analysis of the test or biopsy sample, among others. For example, a user can correspond to a physician, a pathologist, a clinician, or a subject. A user can use one or more user devices 130 to submit one or more requests (e.g., that identify a subject) that a sample be processed by digital pathology image generation subsystem 110 and that a resulting image be processed by SNR/SNCD segmentation subsystem 112, neural cell segmentation and quantification subsystem 114, or other components of system 100, or combinations thereof.

In some embodiments, the biological samples that will be prepared for imaging may be collected from one or more preclinical trials. In one or more examples, the preclinical trials may include procedures to induce dopaminergic neural cell loss in regions of SN. For example, artificial insults may be used, such as injections of pathological proteins, expression of AAV vectors with mutant proteins that lead to PD. Additionally, Transgenic animal models expressing mutant proteins that are linked to PD which can inflict dopaminergic neural cell loss can be also studied. For example, dopaminergic neural cell loss may be induced in animal models, such as mice models, as a measure of a pathological end-point that can be used to measure drug efficacy against PD. The number of subjects in a preclinical trial can vary from study to study. In general, the number of animals studied can be anything between 50 to 1000 animals.

In some embodiments, digital pathology image generation subsystem 110 may be configured to transmit an image produced by the image scanner to user device 130. User device 130 may communicate with SNR/SNCD segmentation subsystem 112, neural cell segmentation and quantification subsystem 114, or other components of computing system 102 to initiate automated processing and analysis of the digital pathology image. In some embodiments, digital pathology image generation subsystem 110 may be configured to provide a digital pathology image (e.g., a whole slide image) to SNR/SNCD segmentation subsystem 112 and/or neural cell segmentation and quantification subsystem 114.

In some embodiments, a trained pathologist may manually annotate one or more images to indicate regions of SNR and/or regions of SNCD within the images. In one or more examples, the trained pathologist may generate first segmentation maps for the images. The first segmentation maps may be bit-masks, or “masks.” In some embodiments, the first segmentation maps may comprise pixel-wise labels indicating whether a corresponding pixel of image depicts a region of SNR, a region of SNCD, or a region of non-SN brain tissue. In one or more examples, the first segmentation maps may include an SNR bit mask used to indicate which pixels of an image depict regions of SNR. The pixel-wise labels may be binary labels where a bit may be assigned a first value (e.g., logical 0) if the corresponding pixel depicts a region of SNR or a second value (e.g., a logical 1) if the corresponding pixel does not depict a region of SNR. In one or more examples, the first segmentation maps may include an SNCD bit-mask used to indicate which pixels of an image depict regions of SNCD. The pixel-wise labels may be binary labels where a bit may be assigned a first value (e.g., logical 0) if the corresponding pixel depicts a region of SNCD or a second value (e.g., a logical 1) if the corresponding pixel does not depict a region of SNCD. In some embodiments, the images may be annotated to include outlines of the regions of SNR and the regions of SNCD. The first segmentation maps and/or annotations may be stored in association with the images in image database 142 and/or training data database 144.

In some embodiments, a trained pathologist may manually annotate one or more images to indicate dopaminergic neural cells within one or more ROIs (e.g., regions of SNR and/or regions of SNCD) within the images. In one or more examples, the trained pathologist may generate second segmentation maps for the images. The second segmentation maps may also be bit-masks, or “masks.” In some embodiments, the second segmentation maps may comprise pixel-wise labels indicating whether a corresponding pixel of image depicts a portion of a dopaminergic neural cell. For instance, the pixel-wise labels may be binary labels where a bit may be assigned a first value (e.g., logical 0) if the corresponding pixel depicts a portion of a dopaminergic neural cell or a second value (e.g., a logical 1) if the corresponding pixel does not depict a portion of a dopaminergic neural cell.

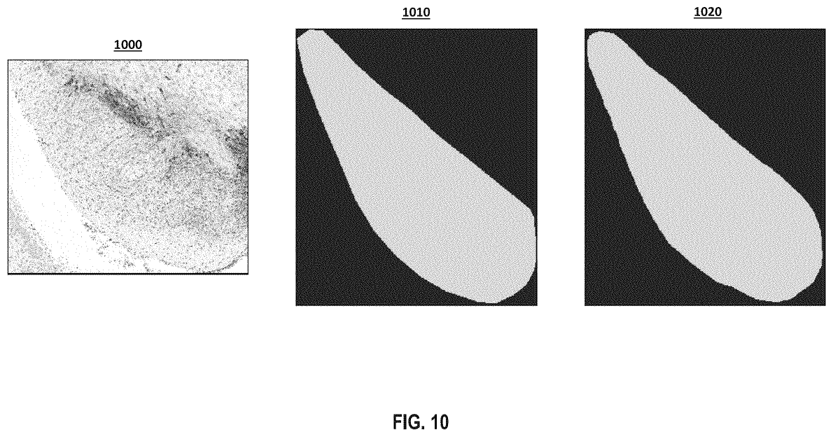

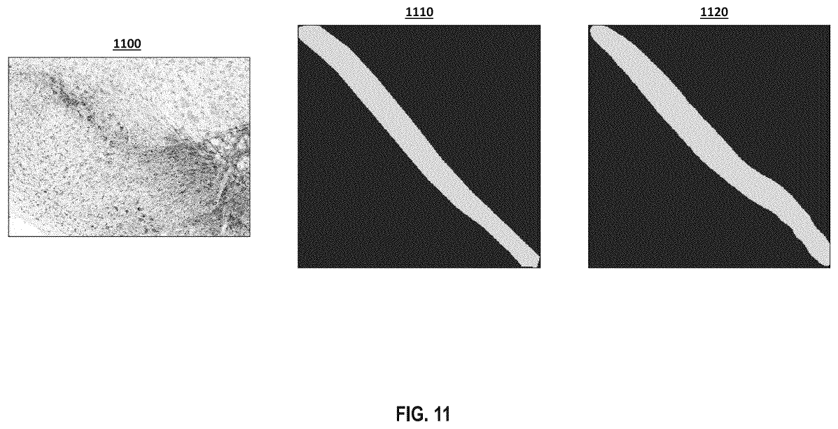

SNR/SNCD segmentation subsystem 112 may be configured to identify regions of substantia nigra reticulata (SNR) and regions of substantia nigra compacta dorsal (SNCD) in images of a subject exhibiting dopaminergic neural cell loss. For example, the subject may be diagnosed with Parkinson's disease (PD), which can cause dopaminergic neural cell loss in regions of SN. In some embodiments, SNR/SNCD segmentation subsystem 112 may be configured to receive an image depicting a section of a brain including substantia nigra (SN) of the subject. For example, with reference to FIG. 10, SNR/SNCD segmentation subsystem 112 may receive an image 1000 depicting a section of a brain including SN of the subject. As another example, with reference to FIG. 11, SNR/SNCD segmentation subsystem 112 may receive an image 1100 depicting a section of a brain including SN of a subject. In some embodiments, image 1000 and image 1100 may be the same or similar. In some embodiments, image 1000 and image 1100 may be derived from a whole slide image. For example, image 1000 may correspond to a first portion of a whole slide image of a brain of a subject, and image 1100 may correspond to a second portion of the whole slide image. In one or more examples, image 1000 and image 1100 include one or more overlapping pixels. In one or more examples, image 1000 and image 1100 have no overlapping pixels. In one or more examples, the image received by SNR/SNCD segmentation subsystem 112 may comprise a whole slide image, or a portion thereof, of a section of a brain of a subject.

In some embodiments, SNR/SNCD segmentation subsystem 112 may be configured to obtain a segmentation map of the image by inputting the image into a trained machine learning model. For example, with reference again to FIG. 10, SNR/SNCD segmentation subsystem 112 may input image 1000 into a trained machine learning model to obtain segmentation map 1020. As another example, with reference again to FIG. 11, SNR/SNCD segmentation subsystem 112 may input image 1000 into a trained machine learning model to obtain segmentation map 1120. In one or more examples, the segmentation map (e.g., segmentation map 1020, segmentation map 1120) may comprise a plurality of pixel-wise labels. Each pixel-wise label may indicate that a corresponding pixel of the image (e.g., image 1000, image 1100) comprises a portion of one or more regions of SNR, a portion of one or more regions of SNCD, or a portion of non-SN brain tissue. For example, coordinates of segmentation map 1020 that are highlighted “yellow” may indicate that a corresponding pixel within image 1000 depicts at least a portion of a region of SNR. As another example, coordinates of segmentation map 1120 that are highlighted “yellow” may indicate that a corresponding pixel within image 1100 depicts at least a portion of a region of SNCD.

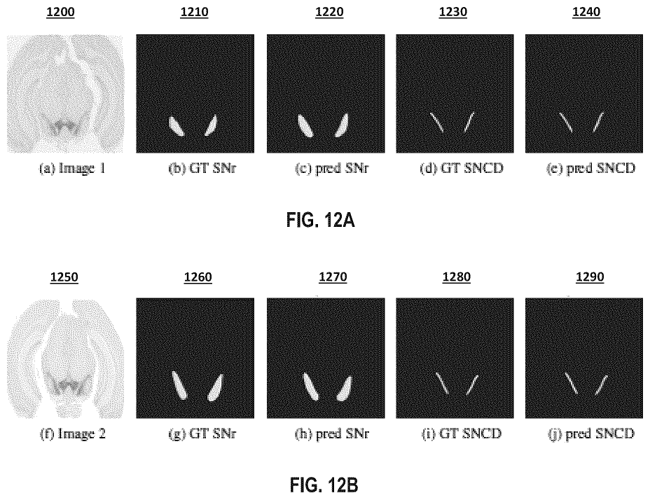

In some embodiments, SNR/SNCD segmentation subsystem 112 may be configured to identify one or more regions of SNR and one or more regions of SNCD based on the segmentation map of the image. For example, as seen with reference to FIGS. 12A and 12B, images 1200 and 1250 may be input to a trained machine learning model to obtain SNR segmentation maps 1220 and 1270, respectively, indicating pixels of images 1200 and 1250 that correspond to at least a portion of a region of SNR. Similarly, images 1200 and 1250 may be input to the trained machine learning model to obtain SNCD segmentation maps 1240 and 1290, respectively, indicating pixels of images 1200 and 1250 that correspond to at least a portion of a region of SNCD. Also depicted in FIGS. 12A-12B are precomputed SNR segmentation maps 1210 and 1260 and precomputed SNCD segmentation maps 1230 and 1280. Precomputed SNR segmentation maps 1210 and 1260 and precomputed SNCD segmentation maps 1230 and 1280 may be generated by a trained pathologist based on images 1200 and 1250 to indicate regions of SNR and SNCD, respectively. Precomputed SNR segmentation maps 1210 and 1260 and precomputed SNCD segmentation maps 1230 and 1280 may be used as ground truths for determining an accuracy of the trained model and further adjusting one or more hyperparameters of the model to improve the model's ability to generate SNR and SNCD segmentation maps.

In FIG. 10, FIG. 11, and FIGS. 12A-12B, coordinates within the segmentation maps highlighted in “purple” may correspond to non-SN brain tissue.

In one or more examples, the section of the brain depicted by the image may be stained with a stain highlighting SN. For example, the stain may be a tyrosine hydroxylase enzyme (TH). TH may be used because it is an indicator of dopaminergic neuron viability. As seen, for example, within image 1000, a TH stain applied to the biological sample depicted thereby may cause dopaminergic neural cells contained therein to highlight brown. In some embodiments, SNR/SNCD segmentation subsystem 112 may be configured to generate segmentation maps by determining each of the pixel-wise labels based on an intensity of one or more stains applied to a biological sample of the section of the brain. The stains may be configured to highlight the regions of SNR, the regions of SNCD, and the non-SN brain tissue within the biological sample. For example, the stain may be a TH stain configured to highlight dopaminergic neural cells. In one or more examples, each pixel-wise label may indicate whether a corresponding pixel in the image depicts at least one of the regions of SNR, at least one of the regions of SNCD, or the non-SN brain tissue.

In some embodiments, SNR/SNCD segmentation subsystem 112 may be configured to calculate an optical density of dopaminergic neural cells within the regions of SNR and the regions of SNCD based on an expression level of the stain within the image. For example, the stain may cause a dopaminergic neuron to turn a particular color (e.g., brown). The intensity of that color can be quantified and used as an indication of the likelihood that a corresponding pixel of the image depicts a dopaminergic neuron. In one or more examples, the intensity of the pixel may be compared to a threshold pixel intensity. If the intensity of the pixel is greater than or equal to the threshold pixel intensity, that pixel may be classified as depicting at least a portion of a dopaminergic neuron. In some embodiments, SNR/SNCD segmentation subsystem 112 may be further configured to predict a health state of the dopaminergic neural cells within the regions of SNR and the regions of SNCD based on the calculated optical density. For example, the health status of dopaminergic neural cells may relate to the intensity of the TH stain. The TH stain is absorbed by dopaminergic cells to cause them to express as a certain color. The greater the intensity of that color within, the healthier (and abundant) the dopaminergic neural cells are.

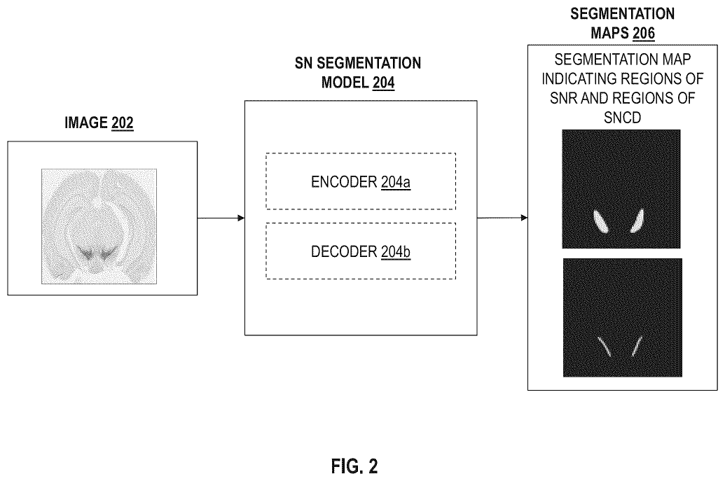

SNR/SNCD segmentation subsystem 112 may obtain the SNR segmentation map and the SNCD segmentation maps from a trained machine learning model. As an example, with reference to FIG. 2, SNR/SNCD segmentation subsystem 112 may be configured to input image 202 into SN segmentation model 204, which may generate and output one or more segmentation maps 206. In one or more examples, SN segmentation model 204 may be configured to output an SNR segmentation map indicating one or more regions of SNR within image 202 and an SNCD segmentation map indicating one or more regions of SNCD within image 202. In one or more examples, SN segmentation model 204 may be configured to output a single segmentation map indicating one or more regions of SNR and/or one or more regions of SNCD within image 202.

In some embodiments, SNR/SNCD segmentation subsystem 112 may be configured to train a machine learning model, such as SN segmentation model 204, to generate segmentation maps 206 based in input image 202. In some embodiments, the trained machine learning model may be implemented using an encoder-decoder architecture comprising an encoder and a decoder. For example, SN segmentation model 204 may include an encoder 204a and a decoder 204b. In one or more examples, encoder 204a may be configured to extract one or more features from an image (e.g., a training image, an input image). In one or more examples, decoder 204b may be configured to classify one or more pixels of image 202. For example, decoder 204b may classify a pixel of image 202 as depicting at least a portion of a region of SNR, at least a portion of a region of SNCD, or at least a portion of non-SN brain tissue.

To train machine learning models to generate segmentation maps indicating regions of SNR and regions of SNCD within images depicting brains, the training images should include images pre-determined to include regions of SNR and regions of SNCD. However, large databases of such images do not exist due to the complexities with which it takes to develop. Therefore, one commonly used tool is to use transfer learning. In transfer learning, a model can be trained on a large corpus of natural images, such as the ImageNet dataset, and then fine-tuned on a smaller, task-specific set of images. As a result, pre-trained networks can be used to acquire some of the fundamental parameters. One example network that may be implemented as encoder 204a is Efficient-Net, which may perform feature extraction. For example, the architecture used for encoder 204a may include a plurality of stages i with \hat{L}ᵢ layers having input resolution (Ĥᵢ, Ŵᵢ) and output channels Ĉᵢ. Table 1 below illustrates the example resolutions, operators, channels, layers for each stage.

TABLE 1

| Stage | Operator | Resolution | #Channels | #Layers |
|---|---|---|---|---|
| i | [symbol] | Ĥᵢ × Ŵᵢ | Ĉᵢ | \hat{L}ᵢ |
| 1 | Conv3x3 | 224 × 224 | 32 | 1 |
| 2 | MBConv1, k3x3 | 112 × 112 | 16 | 1 |
| 3 | MBConv6, k3x3 | 112 × 112 | 24 | 2 |
| 4 | MBConv6, k5x5 | 56 × 56 | 40 | 2 |
| 5 | MBConv6, k3x3 | 28 × 28 | 80 | 3 |
| 6 | MBConv6, k5x5 | 14 × 14 | 112 | 3 |
| 7 | MBConv6, k5x5 | 14 × 14 | 192 | 4 |
| 8 | MBConv6, k3x3 | 7 × 7 | 320 | 1 |
| 9 | Conv1x1 & Pooling & FC | 7 × 7 | 1280 | 1 |

EfficientNet uses a compound coefficient to equally scale depth, width, and resolution. As an example, the number of parameters and FLOPs used for the model implemented as encoder 204a may be 30 M and 9.9B, respectively.

In some embodiments, decoder 204b may be configured to perform semantic segmentation. In one or more examples, decoder 204b may be implemented as a U-Net model. Decoder 204b may be configured to generate feature maps. The feature maps generated by encoder 204a may serve as the input to go through up-sampling layers of decoder 204b. As an example, the U-Net model, which may be used for decoder 204b, may include a contracting path and an expansive path. The contracting path follows the typical architecture of a convolutional network, consisting of repeated application of two 3×3 convolutions (unpadded convolutions), each followed by a rectified linear unit (ReLU) and a 2×2 max pooling operation with stride 2 for down-sampling. At each down-sampling step we double the number of feature channels. Every step in the expansive path consists of an up-sampling of the feature map followed by a 2×2 convolution (“up-convolution”) that halves the number of feature channels, a concatenation with the correspondingly cropped feature map from the contracting path, and two 3×3 convolutions, each followed by a ReLU. The cropping is necessary due to the loss of border pixels in every convolution. At the final layer, a 1×1 convolution is used to map each 64-component feature vector to the desired number of classes. In total the network has 23 convolutional layers.

In some embodiments, SN segmentation model 204 may further include a final layer comprising a SoftMax activation function. The reason for this is that the task is to perform multiclass segmentation where the different classes are a region of SNR, a region of SNCD, and a region of non-SN brain tissue.

The training process may use a plurality of training images to obtain the trained machine learning model, which can be deployed as SN segmentation model 204. In one or more examples, each of the training images depicts a section of a brain including SN. Each of the training images may also include, or be associated with, a precomputed segmentation map corresponding to that training image. For example, with reference to FIG. 10, precomputed SNR segmentation map 1010 may correspond to a segmentation map indicating regions of SNR generated by a trained pathologist based on image 1000. Similarly, with reference to FIG. 11, precomputed SNCD segmentation map 1110 may correspond to a segmentation map indicating regions of SNCD generated by the trained pathologist. As yet another example, with reference to FIGS. 12A-12B, precomputed SNR segmentation maps 1210 and 1260 and precomputed SNCD segmentation maps 1230 and 1280 may be generated by a trained pathologist based on image 1200 and image 1250 to indicate regions of SNR and SNCD, respectively. Precomputed SNR segmentation maps 1210 and 1260 and precomputed SNCD segmentation maps 1230 and 1280 may be used as ground truths for determining an accuracy of the trained model and further adjusting one or more hyperparameters of the model to improve the model's ability to generate SNR and SNCD segmentation maps.

In some embodiments, SNR/SNCD segmentation subsystem 112 may be configured to train the machine learning model by retrieving a plurality of images each depicting a section of a brain including SN and performing one or more image transformation operations to each of the images to obtain the training images. In one or more examples, the image transformation operations comprise at least one of a rotation operation, a horizontal flip operation, a vertical flip operation, a random 90-degree rotation operation, a transposition operation, an elastic transformation operation, cropping, or a Gaussian noise addition operation, or other image transformation operations.

In some embodiments, SNR/SNCD segmentation subsystem 112 may be configured to adjust a size of one or more of the training images such that each of the training images has a same size. For example, a whole slide image may be 100,000×100,000 pixels, making it difficult and time-consuming to use for training. Thus, the size of the whole slide image may be adjusted (e.g., cropping, zooming, etc.) to a smaller size. In one or more examples, the size of each of the training images is 1024× 1024 pixels.

In some embodiments, SNR/SNCD segmentation subsystem 112 may be configured to train a machine learning model based on the plurality of training images to obtain the trained machine learning model, for example SN segmentation model 204. Training the machine learning model may include, for each of the training images, extracting one or more features from the training image. In one or more examples, a feature vector representing the training image may be generated based on the one or more extracted features. One or more pixels of the training image may be classified, based on the feature vector, as representing a portion of the regions of SNR, a portion of the regions of SNCD, or a portion of non-SN brain tissue. In one or more examples, a segmentation map for the training image may be generated based on the classification of each pixel. In some embodiments, the segmentation maps generated by the trained machine learning mode, for example, SN segmentation model 204, may be bit-masks, where each bit corresponds to a pixel from the input image, and the value of the bit depends on the classification. For example, for the SNR segmentation map, each bit may correspond to a pixel from the input image and may have a value indicating whether that pixel depicts a portion of a region of SNR or a portion of non-SN brain tissue. As another example, for the SNCD segmentation map, each bit may correspond to a pixel from the input image and may have a value indicating whether that pixel depicts a portion of a region of SNCD or a portion of non-SN brain tissue. In some embodiments, a single segmentation map may be generated that includes bits that can have a first value indicating that a corresponding pixel of an input image depicts a region of SNR, a region of SNCD, or non-SN brain tissue.

In some embodiments, for each of the plurality of training images, SNR/SNCD segmentation subsystem 112 may be configured to calculate a similarity score between the segmentation map generated for the training image and the precomputed segmentation map for the training image. For example, with reference again to FIG. 10, a similarity score may be computed based on predicted SNR segmentation map 1020 and precomputed SNR segmentation map 1010. As another example, with reference again to FIG. 11, a similarity score may be computed based on predicted SNCD segmentation map 1120 and precomputed SNCD segmentation map 1110. As still yet another example, with reference again to FIG. 12A, a similarity score may be computed based on predicted SNR segmentation map 1220 and precomputed SNR segmentation map 1210 and a similarity score may be computed based on predicted SNCD segmentation map 1240 and precomputed SNCD segmentation map 1230. As still yet another example, with reference again to FIG. 12B, a similarity score may be computed based on predicted SNR segmentation map 1270 and precomputed SNR segmentation map 1260 and a similarity score may be computed based on predicted SNCD segmentation map 1290 and precomputed SNCD segmentation map 1280.

Based on the similarity score(s), one or more hyperparameters of the trained machine learning model, for example SN segmentation model 204 may be adjusted. The adjustments to the hyperparameters of the trained machine learning model may function to enhance a similarity between the generated segmentation map and the precomputed segmentation map. In some embodiments, one or more loss functions may be used to compute the similarity. For example, the loss functions may be Dice, Jaccard, or categorical cross-entropy, however alternative loss functions may be used. As another example, the optimizers used may be the Adam optimizer, Stochastic Gradient Descent, or other optimizers.

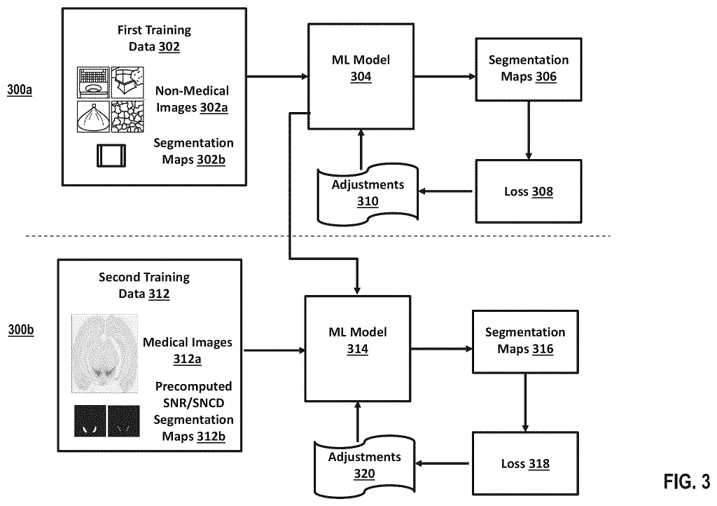

In some embodiments, SNR/SNCD segmentation subsystem 112 may be configured to train SN segmentation model 204 using two training steps. For example, as seen with reference to FIG. 3, SNR/SNCD segmentation subsystem 112 may be configured to perform a first training step 300a to a machine learning model 304 to obtain a train machine learning model 314, with which a second training step 300b to obtain the trained machine learning model (e.g., SN segmentation model 204). In one or more examples, first training step 300a performed to machine learning model 304 may be based on first training data 302 comprising a plurality of non-medical images 302a. First training data 302 may also include precomputed classifications and/or segmentation maps for the non-medical images. The classifications may indicate a class of object or objects depicted by the image and the segmentation map may indicate which pixels represented a particular class of object within the image. For example, the non-medical images may comprise natural images, such as those included within the ImageNet dataset.

In some embodiments, first training step 300a may include non-medical images 302a of first training data 302 being input to ML model 304 to obtain predicted segmentation maps 306. Predicted segmentation maps 306 may be compared to precomputed segmentation maps included in first training data 302 to compute loss 308. In one or more examples, loss 308 may be computed by calculating a Dice function loss, however alternative loss functions may be used. Based on loss 308, SNR/SNCD segmentation subsystem 112 may cause adjustments 310 to be made to ML model 304. SNR/SNCD segmentation subsystem 112 may be configured to repeat first training step 300a a predefined number of times or until an accuracy of ML model 304 satisfies a threshold accuracy.

In some embodiments, first training data 302 may include sets of non-medical images 302a and segmentation maps 302b separated into training, validation, and testing sets. Thus, ML model 304 may be considered “trained,” or finished with first training step 300a, when ML model 304 is able to predict the segmentation map for a non-medical image of the test set with an accuracy greater than or equal to the threshold accuracy.

In one or more examples, second training step 300b performed to machine learning model 314 may be based on second training data 312 comprising (i) a plurality of medical images 312a depicting sections of the brain including SN and (ii) a precomputed segmentation map 312b for each of medical images 312a indicating regions of SNR/SNCD. In some embodiments, ML model 314 may comprise the “trained” version of ML model 304. In other words, once ML model 304 has been trained using non-medical images 302a, transfer learning can be used to tune hyperparameters of ML model 314, which can be trained on medical images 312a.

In some embodiments, second training step 300b may include medical images 312a of second training data 312 being input to ML model 314 to obtain predicted SNR/SNCD segmentation maps 316. Predicted SNR/SNCD segmentation maps 306 may be compared to precomputed SNR/SNCD segmentation maps 312b included in second training data 302 to compute loss 318. In one or more examples, loss 318 may be computed by calculating a Dice function loss, however alternative loss functions may be used. Based on loss 318, SNR/SNCD segmentation subsystem 112 may cause adjustments 320 to be made to ML model 314. SNR/SNCD segmentation subsystem 112 may be configured to repeat the second training step 300b a predefined number of times or until an accuracy of ML model 314 satisfies a threshold accuracy.

In some embodiments, second training data 302 may include sets of medical images 312a and precomputed SNR/SNCD segmentation maps 312b separated into training, validation, and testing sets. Thus, ML model 314 may be considered “trained,” or finished with second training step 300b, when ML model 314 is able to predict the segmentation map (e.g., SNR segmentation map, SNCD segmentation map) for medical image of the test set with an accuracy greater than or equal to the threshold accuracy.

In some embodiments, precomputed segmentation maps 312b for each of medical images 312a may comprise a plurality of pixel-wise labels. In one or more examples, each pixel-wise label may indicate whether a corresponding pixel of the image of medical images 312a comprises a portion of one or more regions of SNR, a portion of one or more regions of SNCD, or a portion of non-SN brain tissue. For example, if an SNR segmentation map and an SNCD segmentation map are produced, then each pixel-wise label of the SNR segmentation map can indicate whether a corresponding pixel from an input image represents a portion of a region of SNR or a portion of non-SN brain tissue, and each pixel-wise label of the SNCD segmentation map can indicate whether a corresponding pixel from an input image represents a portion of a region of SNCD or a portion of non-SN brain tissue. In some embodiments, where a single segmentation map may be output, the pixel-wise label may indicate whether a corresponding pixel in the input image represents a portion of a region of SNR, a portion of a region of SNCD, or a portion of non-SN background tissue.

In some embodiments, second training step 300b may be performed after first training step 300a.

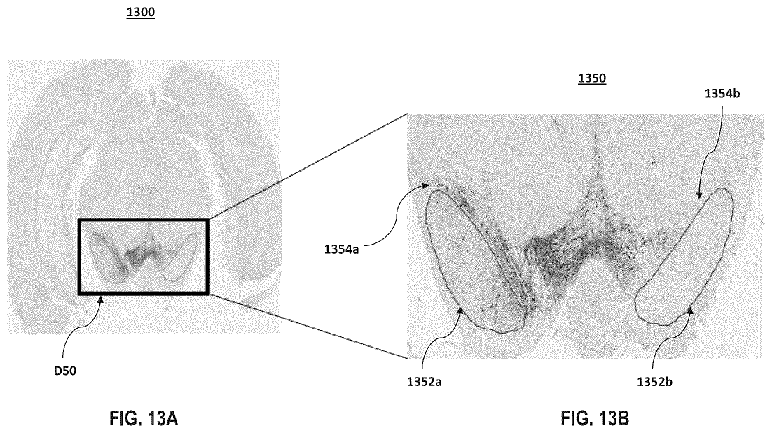

In some embodiments, SNR/SNCD segmentation subsystem 112 may be configured to generate an annotated version of the image. The annotated version of the image may include a first visual indicator defining the regions of SNR within the image and a second visual indicator defining the regions of SNCD within the image. For example, as seen with reference to FIGS. 13A-13B, image 1300 depicts a brain of a subject and image 1350 depicts a zoomed-in image 1350 of image 1300 including annotations 1352a-1352b and 1354a-1354b indicating a location of one or more regions of SNR and one or more regions of SNCD, respectively, for either brain hemisphere. As seen in image 1350, annotations 1352a-1352b and 1354a-1354b may outline the regions of SNR and SNCD, respectively in “red” and “yellow.”

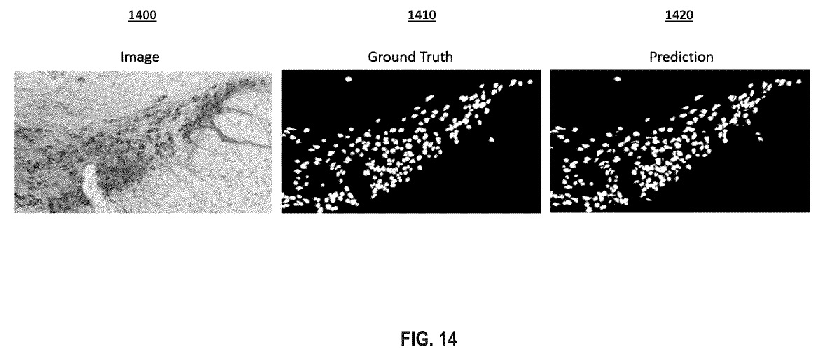

Returning to FIG. 1, neural cell segmentation and quantification subsystem 114 may be configured to determine a number of dopaminergic neural cells within an image depicting a section of a brain of a subject exhibiting dopaminergic neural cell loss. For example, the subject may be diagnosed with Parkinson's disease (PD), which can cause dopaminergic neural cell loss in regions of SN. In some embodiments, neural cell segmentation and quantification subsystem 114 will receive an image depicting a section of a brain of a subject. In one or more examples, the subject may be diagnosed with a disease. For example, the subject may be diagnosed with Parkinson's disease (PD). As an example, with reference to FIG. 14, image 1400 depicts a section of a brain of a subject. In particular, image 1400 may include a depiction of one or more ROIs where dopaminergic neural cells are located within the brain. For example, image 1400 may depict at least a portion of a region of SNR and/or at least a portion of a region of SNCD. In some embodiments, image 1400 may be derived from a whole slide image.

In some embodiments, neural cell segmentation and quantification subsystem 114 may be configured to divide the image into a plurality of patches. In one or more examples, the patches may be non-overlapping. In one or more examples, the patches may have a size of 512× 512 pixels.

In some embodiments, neural cell segmentation and quantification subsystem 114 may be configured to generate, using a trained machine learning model, a segmentation map for each of the patches. The trained machine learning model implemented by neural cell segmentation and quantification subsystem 114 may be a separate model than that implemented by SNR/SNCD segmentation subsystem 112. Similarly, the segmentation map generated by neural cell segmentation and quantification subsystem 114 may be a different segmentation map than that produced by SNR/SNCD segmentation subsystem 112. In one or more examples, the segmentation map generated by neural cell segmentation and quantification subsystem 114 may comprise a plurality of pixel-wise labels. In one or more examples, each label may indicate whether a corresponding pixel in the image is classified as depicting dopaminergic neural cells or neural background tissue. As an example, as seen by FIG. 14, segmentation map 1420 may indicate which pixels from image 1400 depict dopaminergic neural cells and which pixels form image 1400 depict neural background tissue. In some examples, each pixel-wise label may comprise a first value or a second value, where the first value (e.g., a logical 0) indicates that a corresponding pixel from image 1400 depicts at least a portion of a dopaminergic neural cell and the second value (e.g., logical 1) indicates that a corresponding pixel from image 1400 depicts neural background tissue.

In some embodiments, neural cell segmentation and quantification subsystem 114 may be configured to determine a number of dopaminergic neural cells within the image based on the segmentation map generated for each of the plurality of patches. For example, neural cell segmentation and quantification subsystem 114 may determine a quantity of dopaminergic neural cells depicted within each patch (e.g., image 1400 of FIG. 14) based on the segmentation map generated for that patch (e.g., segmentation map 1420). In some embodiments, neural cell segmentation and quantification subsystem 114 may identify clusters of dopaminergic neural cells. For each cluster, neural cell segmentation and quantification subsystem 114 may determine a number of dopaminergic cells included within that cluster. In one or more examples, neural cell segmentation and quantification subsystem 114 may determine whether the cluster depicts multiple dopaminergic cells based on an average size of a dopaminergic neural cell. The average size of the dopaminergic neural cell may be calculated based on training data used to train a machine learning model implemented by neural cell segmentation and quantification subsystem 114 to generate the segmentation maps.

In some embodiments, neural cell segmentation and quantification subsystem 114 may further be configured to determine each of the pixel-wise labels based on an intensity of a stain applied to a biological sample of the section of the brain. In one or more examples, the stain is selected such that it highlights dopaminergic neural cells within a biological sample. For example, the section of the brain depicted by the image may be stained with a stain highlighting SN. For example, the stain may be a tyrosine hydroxylase enzyme (TH). TH may be used because it is an indicator of dopaminergic neuron viability. As seen, for example, within image 1400, a TH stain applied to the biological sample depicted thereby may cause dopaminergic neural cells contained therein to highlight brown. In some embodiments, neural cell segmentation and quantification subsystem 114 may be configured to generate the segmentation maps for each patch by determining each of the pixel-wise labels based on an intensity of one or more stains applied to a biological sample of the section of the brain. For example, the stain may be a TH stain configured to highlight dopaminergic neural cells. In one or more examples, each pixel-wise label may indicate whether a corresponding pixel in the image depicts at least a portion of a dopaminergic neural cell (e.g., a single cell or a cluster of cells) or neural background tissue. As an example, with reference again to FIG. 14, the pixel-wise labels included in predicted segmentation map 1420 may indicate whether the corresponding pixel of image 1400 depicts a dopaminergic neural cell or neural background tissue. In some embodiments, neural cell segmentation and quantification subsystem 114 may determine whether the intensity of the stain of a given pixel is greater than or equal to a threshold intensity. If so, neural cell segmentation and quantification subsystem 114 may classify that pixel as depicting a dopaminergic neural cell and assign a first value (e.g., logical 0) to the corresponding pixel-wise label. If not, neural cell segmentation and quantification subsystem 114 may classify that pixel as depicting neural background tissue and assign a second value (e.g., logical 1) to the corresponding pixel-wise label. In the illustrative example of FIG. 14, pixel-wise labels having the first value may be colored “white” within predicted segmentation map 1420 and pixel-wise labels having the second value may be colored “black” within predicted segmentation map 1420.

In some embodiments, neural cell segmentation and quantification subsystem 114 may be configured to determine a health state of the dopaminergic neural cells based on the intensity of the stain expressed by each pixel of the image classified as depicting a dopaminergic neural cell. Some embodiments, neural cell segmentation and quantification subsystem 114 may be further configured to predict a health state of the dopaminergic neural cells based on the intensity of the TH stain. The TH stain is absorbed by dopaminergic cells to cause them to express as a certain color. The greater the intensity of that color within, the healthier (and abundant) the dopaminergic neural cells may be.

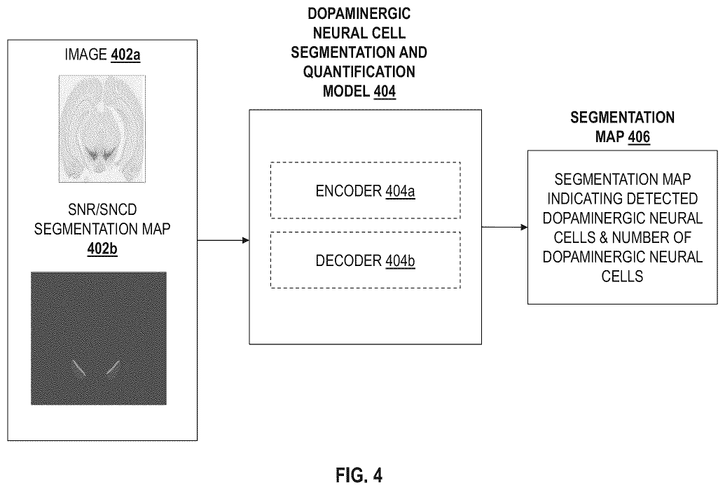

In some embodiments, neural cell segmentation and quantification subsystem 114 may further be configured to train a machine learning model to recognize dopaminergic neural cells within an input image to obtain the trained machine learning model. As an example, with reference to FIG. 4, neural cell segmentation and quantification subsystem 114 may be configured to input image 402a into a dopaminergic neural cell segmentation and quantification model 404 to obtain segmentation map 406. In one or more examples, segmentation map 406 may indicate locations of dopaminergic neural cells detected within image 402a as well as a quantity of dopaminergic neural cells present within image 402a. In some embodiments, one or more SNR/SNCD segmentation maps 402b may be input to dopaminergic neural cell segmentation and quantification model 404. For example, a predicted SNR segmentation map (e.g., predicted SNR segmentation map 1020 of FIG. 10) and/or a predicted SNCD segmentation map (e.g., predicted SNCD segmentation map 1120 of FIG. 11) may be input to dopaminergic neural cell segmentation and quantification model 404 along with a corresponding image 402a. SNR/SNCD segmentation maps 402b may indicate ROIs where dopaminergic neural cell segmentation and quantification model 404 should focus on when attempting to detect and quantify dopaminergic neural cells within image 402a.

In some embodiments, dopaminergic neural cell segmentation and quantification model 404 may be implemented as an encoder-decoder model including an encoder 404a and a decoder 404b. In some examples, dopaminergic neural cell segmentation and quantification model 404 may be implemented as a U-Net model. The U-Net model, as described above, may include a contracting path and an expansive path. The contracting path follows the typical architecture of a convolutional network, consisting of repeated application of two 3×3 convolutions (unpadded convolutions), each followed by a rectified linear unit (ReLU) and a 2×2 max pooling operation with stride 2 for down-sampling. At each down-sampling step we double the number of feature channels. Every step in the expansive path consists of an up-sampling of the feature map followed by a 2×2 convolution (“up-convolution”) that halves the number of feature channels, a concatenation with the correspondingly cropped feature map from the contracting path, and two 3×3 convolutions, each followed by a ReLU. The cropping is necessary due to the loss of border pixels in every convolution. At the final layer, a 1×1 convolution is used to map each 64-component feature vector to the desired number of classes. In total the network has 23 convolutional layers. In some embodiments, encoder 404a may be implemented using a ResNet model. For example, encoder 404a may be implemented using ResNet-50. In some embodiments, encoder 404a of dopaminergic neural cell segmentation and quantification model 404 may be mathematically represented by f_{θ} and decoder 404b may be mathematically represented by g_{θ}.

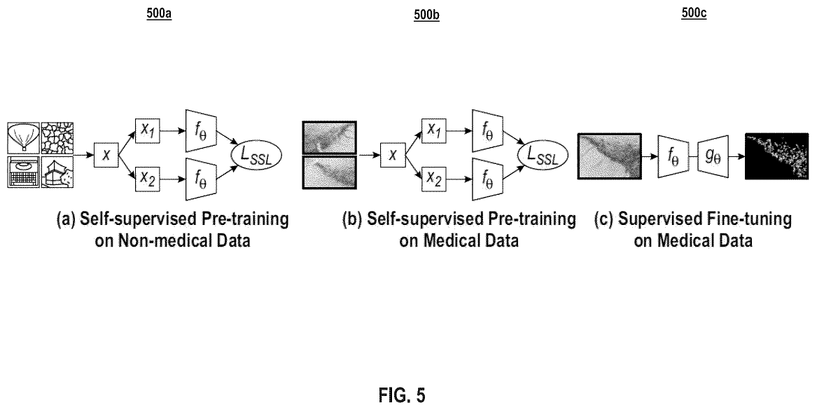

In some embodiments, neural cell segmentation and quantification subsystem 114 may further be configured to train dopaminergic neural cell segmentation and quantification model 404 using a multi-step training process. For example, with reference to FIG. 5, training dopaminergic neural cell segmentation and quantification model 404 may include a first training step 500a, a second training step 500b, and a third training step 500c. In one or more examples, first training step 500a may comprise performing a first self-supervised learning (SSL) step. In first training step 500a, an encoder may be trained on first training data to obtain a first trained encoder. The first training data may include a plurality of non-medical images. In one or more examples, the non-medical images may comprise natural images, such as those included in the ImageNet dataset. In one or more examples, second training step 500b may comprise performing a second SSL step to the first trained encoder based on second training data to obtain a second trained encoder. The second training data may include a first plurality of domain-specific images (e.g., medical images depicting a section of a brain including dopaminergic neural cells). In some embodiments, first training step 500a and second training step 500b may comprise training using discrimination approaches, such as Barlow Twins, however other self-supervised techniques, including but not limited to BYOL, DINO, etc., may be used at first training step 500a and/or second training step 500b. In one or more examples, third training step 500c may include a supervised learning process performed using third training data, where the third training data may include in-domain images (e.g., medical images depicting a section of a brain including dopaminergic neural cells). In some embodiments, the first training data, second training data, and third training data used during first training step 500a, second training step 500b, and third training step 500c may also include ground truth classifications and/or segmentation maps. For example, the second training data used during second training step 500b may include precomputed segmentation maps indicating which pixels of the input image depict a portion of a dopaminergic neural cell and which pixels of the input image depict a portion of neural background tissue. As an example, with reference to FIG. 14, precomputed segmentation map 1410 may be included with image 1400 if used as training data. Predicted segmentation map 1420 may be compared with precomputed segmentation map 1410, and the comparison may be used to adjust hyperparameters of the model to improve accuracy.

In some embodiments, the second training data used during second training step 500b and the third training data used during third training step 500c may include indications of one or more ROIs for the model to focus on. In particular, the ROIs may indicate which portions of the input image should be focused on to detect dopaminergic neural cells. As an example, SNR/SNCD segmentation maps (e.g., SNR/SNCD segmentation maps 402b) indicating regions of SNR and/or regions of SNCD may be included in the second and third training data. In some embodiments, the first training data used during first training step 500a may also include indications of ROIs for the model to focus on and/or predetermined classifications of objects depicted by the non-medical images.

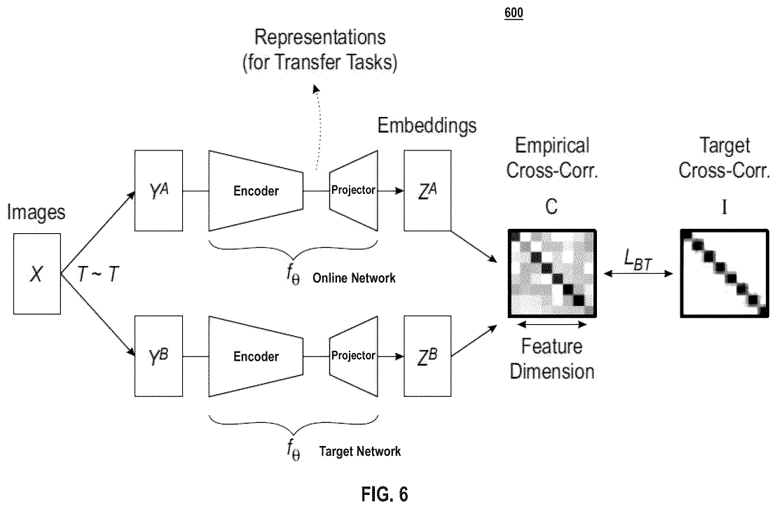

In SSL, a model can be trained using two similarly configured networks: an “online” network and a “target” network that interact and learn from one another. In some embodiments, the online and target networks may be implemented using the same architecture. For example, the online and target networks may be implemented using ResNet-50. As mentioned above, one example SSL technique comprises the Barlow Twins SSL approach. As seen in FIG. 6, for example, SSL approach 600 may include two networks formed of two separate, but similarly configured, components: an encoder and a projector. Each encoder is configured to generate a representation of an input image and project that representation into an embedding space to obtain an output embedding. For example, for a given image X, augmented views Y^{A} and Y^{B} of image X can be created. In one or more examples, image X may comprise a patch of an original image input to the machine learning model. For instance, image X may comprise a patch derived from a whole slide image of a section of a subject's brain (e.g., image 402a of FIG. 4). In some embodiments, neural cell segmentation and quantification subsystem 114 may be configured to generate first augmented view Y^{A} and second augmented view Y^{B} by applying one or more image transformation operations T to image X. In one or more examples, image transformation operations T may include a flip operation, a rotation operation, an RGB shift operation, a blurring operation, a Gaussian noise augmentation operation, a cropping operation, a random resizing operation, or other image transformation operations, or combinations thereof.

In some embodiments, the online network and the target network may both be implemented using an encoder and a projector. For example, the encoder may be a standard ResNet-50 encoder and the projector may be a three-layer MLP projection head.

In some embodiments, the online network may be configured to generate a first representation Z^{A} and the target network may be configured to generate a second representation Z^{B}. In one or more examples, first representation Z^{A} and second representation Z^{B} may be embeddings. Mathematically first representation Z^{A} and second representation Z^{B} may be expressed as:

$${Z}^{A} = {f}_{θ} ( T ( {Y}^{A} ) ) , and$$

$${Z}^{B} = {f}_{θ} ( T ( {Y}^{B} ) ) .$$

In some embodiments, SSL approach 600 may comprise the online network, generating first representation Z^{A}, being trained using first augmented version Y^{A} of image X to predict the target network representation Z^{B} of second augmented version Y^{B} of image X. The rationale behind this process is that the representation of one augmented view of an image should be predictive of the representation of a different augmented view of that same image.

In some embodiments, SSL approach may include a loss computation portion where a difference between first representation Z^{A} and second representation first representation Z^{B} is calculated. In one or more examples, the difference between first representation Z^{A} and second representation first representation Z^{B} may comprise neural cell segmentation and quantification subsystem 114 computing a cross-correlation matrix. For example, the loss function may be represented as:

$${ℒ}_{SSL} = {\sum }_{i} {( 1 - {𝒞}_{ii} )}^{2} + λ {\sum }_{i} {\sum }_{i \neq j} {𝒞}_{ij}^{2} ,$$

Where C is the cross-correlation matrix between first representation Z^{A} and second representation first representation Z^{B} along the batch dimension. The coefficient λ may identify the weight of each loss term. In some embodiments, SSL approach 600 may be designed such that the loss is minimized. In some examples, minimizing the loss function may comprise making the cross-correlation matrix as close as possible to the identity matrix. In particular, by equating the diagonal elements of C to 1 and the off-diagonal elements of C to 0, the learned representation will be invariant to image distortions and the different elements of the representation will be decorrelated such that the output units contain non-redundant information about the input images.

In one or more examples, neural cell segmentation and quantification subsystem 114 may be configured to adjust one or more of the first plurality of hyperparameters of the online network to minimize off-diagonal elements of the cross-correlation matrix and normalize diagonal elements of the cross-correlation matrix. In some embodiments, the hyperparameters of the target network may be updated based on a moving average, an exponential, or another modifier, being applied to the values of the hyperparameters of the online network.

Returning to FIG. 5, first training step 500a may include performing SSL to an encoder (e.g., f_{θ}) using non-medical images, such as the ImageNet dataset. The images may be split, randomly, into training, validation, and test sets (e.g., 70%, 10%, 20% respectively). In some embodiments, first training step 500a may use SSL approach 600 on the non-medical images included in the first training data to train an encoder to obtain a first trained encoder.

In some embodiments, second training step 500b may also use SSL approach 600 on medical images included in the second training data to train the first trained encoder, obtaining a second trained encoder. The second training data may comprise (i) a second plurality of images each depicting a section of a brain comprising dopaminergic neural cells and (ii) predetermined segmentation maps comprising a plurality of pixel-wise labels. Each pixel-wise label may indicate whether a corresponding pixel in a corresponding image of the second plurality of images is classified as depicting a dopaminergic neural cell or neural background tissue. In one or more examples, the second plurality of images may correspond to patches obtained by dividing the input image into a plurality of patches. In some embodiments, the second training data may also include predicted SNR/SNCD segmentation maps generated for the input image. For example, with reference to FIG. 4, SNR/SNCD segmentation map 402b generated by SNR/SNCD segmentation subsystem 112 may be input to neural cell segmentation and quantification subsystem 114 for training dopaminergic neural cell segmentation and quantification model 404 to generate predicted segmentation map 406.

Returning to FIG. 5, in some embodiments, training the machine learning model may further comprise performing a supervised learning step, third training step 500c, to the second trained encoder based on third training data. The third training data may comprise (i) a third plurality of images each depicting a section of a brain comprising dopaminergic neural cells and (ii) predetermined segmentation maps comprising a plurality of pixel-wise labels. Each pixel-wise label may indicate whether a corresponding pixel in a corresponding image of the third plurality of images is classified as depicting a dopaminergic neural cell or neural background tissue. In one or more examples, the third plurality of images may correspond to patches obtained by dividing the input image into a plurality of patches. In some embodiments, the third training data may also include predicted SNR/SNCD segmentation maps generated for the input image. For example, with reference to FIG. 4, SNR/SNCD segmentation map 402b generated by SNR/SNCD segmentation subsystem 112 may be input to neural cell segmentation and quantification subsystem 114 for training dopaminergic neural cell segmentation and quantification model 404 to generate predicted segmentation map 406.

In one or more examples, the third training data may comprise (i) a third plurality of images each depicting a section of a brain comprising at least one region of substantia reticular (SNR) region or at least one region of substantia nigra compacta dorsal (SNCD) region and (ii) second predetermined segmentation maps comprising a plurality of pixel-wise labels. Each pixel-wise label may indicate whether a corresponding pixel in a corresponding image of the third plurality of images is classified as depicting a dopaminergic neural cell or neural background tissue.

In some embodiments, third training step 500c is a supervised learning step where a transfer learning approach is applied. For example, as seen in FIG. 5, the encoder f_{θ} may be used to train a decoder g_{θ} to generate the segmentation map. In some embodiments, third training step 500c may be implemented using the same or similar steps of first training step 300a and/or second training step 300b of FIG. 3. For example, the models of third training step 500c may be trained using the Adam optimizer with a learning rate of 10⁻³, a batch size of 32, and 200 epochs. An early-stop mechanism may be employed to avoid over-fitting. A Dice coefficient loss function may be used for evaluating the accuracy of dopaminergic neural cell segmentation and quantification model 404 of FIG. 4.

In some embodiments, neural cell segmentation and quantification subsystem 114 may be configured to perform first training step 500a (e.g., a first SSL step) for each of the first plurality of non-medical images. In one or more examples, neural cell segmentation and quantification subsystem 114 may be configured to divide each of the non-medical images into a plurality of patches. For each of the patches, neural cell segmentation and quantification subsystem 114 may be configured to generate a first augmented view Y^{A} of a patch X and a second augmented view Y^{B} of patch X. Using a first instance of the encoder comprising a first plurality of hyperparameters, neural cell segmentation and quantification subsystem 114 may be configured to generate a first embedding (e.g., first representation Z^{A}) representing first augmented view Y^{A}. Using a second instance of the encoder comprising a second plurality of hyperparameters, neural cell segmentation and quantification subsystem 114 may be configured to generate a second embedding (e.g., second representation Z^{B}) representing a second augmented view Y^{B}. In some embodiments, neural cell segmentation and quantification subsystem 114 may be further configured to calculate a difference between the first embedding and the second embedding (e.g., cross-correlation loss) and adjust one or more of the first plurality of hyperparameters based on the calculated difference. In some embodiments, neural cell segmentation and quantification subsystem 114 may be configured to adjust the second plurality of hyperparameters of the target network based on the adjustments made to the one or more of the first plurality of hyperparameters of the online network. For example, the values of the hyperparameters of the target network may be updated using a moving average of the values of the hyperparameters of the online network.

In some embodiments, neural cell segmentation and quantification subsystem 114 may be configured to perform a second training step 500b (e.g., the second SSL step) for each of the second plurality of images included in the second training data. In one or more examples, these images may comprise medical images. In particular, the medical images may include images depicting a section or sections of a brain comprising dopaminergic neural cells. In one or more examples, neural cell segmentation and quantification subsystem 114 may be configured to divide each image into a plurality of patches. In one or more examples, the patches are non-overlapping. For each of the plurality of patches (e.g., image X), neural cell segmentation and quantification subsystem 114 may be configured to generate a first augmented view (e.g., first augmented view Y^{A}) and a second augmented view (e.g., first augmented view Y^{B}). It should be noted that the representations and patches of second training step 500b differ from those of first training step 500a, and similar notation is used for simplicity. Using a first instance of the first trained encoder (e.g., the online network) comprising a first plurality of hyperparameters, neural cell segmentation and quantification subsystem 114 may be configured to generate a first embedding (e.g., first representation Z^{A}) representing the first augmented view (e.g., first view Y^{A}). Using a second instance of the first trained encoder (e.g., the target network) comprising a second plurality of hyperparameters, neural cell segmentation and quantification subsystem 114 may be configured to generate a second embedding (e.g., second representation Z^{B}) representing the second augmented view (e.g., second augmented view Y^{B}). In some embodiments, neural cell segmentation and quantification subsystem 114 may be further configured to calculate a difference between the first embedding and the second embedding (e.g., cross-correlation loss) and adjust one or more of the first plurality of hyperparameters based on the calculated difference. In some embodiments, neural cell segmentation and quantification subsystem 114 may be configured to adjust the second plurality of hyperparameters of the target network based on the adjustments made to the one or more of the first plurality of hyperparameters of the online network. For example, the values of the hyperparameters of the target network may be updated using a moving average of the values of the hyperparameters of the online network.

In some embodiments, calculating the difference between the first embedding (e.g., first representation Z^{A}) and the second embedding (e.g., second representation Z^{B}) may comprise neural cell segmentation and quantification subsystem 114 computing a cross-correlation matrix based on the first embedding and the second embedding. In one or more examples, neural cell segmentation and quantification subsystem 114 may be configured to adjust the one or more of the first plurality of hyperparameters to minimize off-diagonal elements of the cross-correlation matrix and normalize diagonal elements of the cross-correlation matrix.

In some embodiments, after training steps 500a-500c have been performed, the trained machine learning model may be deployed, or stored in model database 146 for deployment at a later time. The trained machine learning model may, in some examples, comprise dopaminergic neural cell segmentation and quantification model 404 of FIG. 4.

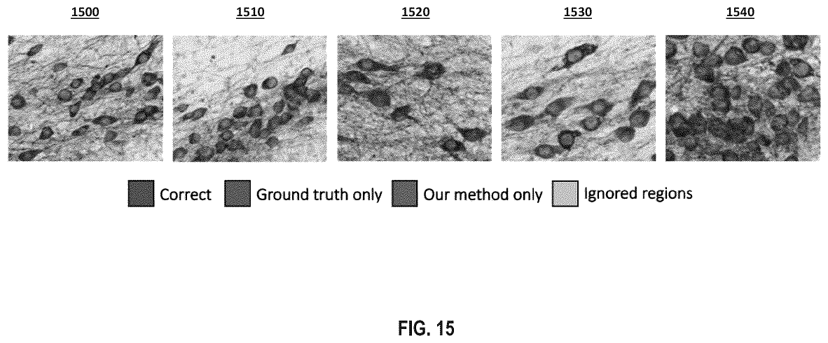

Returning to FIG. 4, dopaminergic neural cell segmentation and quantification model 404 may be configured to detect dopaminergic neural cells within one or more ROIs of an input image. As an example, with reference to FIG. 15, dopaminergic neural cell segmentation and quantification model 404 may detect instances of dopaminergic neural cells within an input image. FIG. 15 includes images 1500-1540. Images 1500-1540 illustrate correctly predicted dopaminergic neural cells 1502, predetermined (e.g., ground truth) dopaminergic neural cells 1504, incorrectly predicted dopaminergic neural cells 1506, and neural background tissue 1508. Correctly predicted dopaminergic neural cells 1502 correspond to locations of dopaminergic neural cells within images 1500-1540 correctly predicted by dopaminergic neural cell segmentation and quantification model 404. Correctly predicted dopaminergic neural cells 1502 are highlighted in “purple” within images 1500-1540. Predetermined dopaminergic neural cells 1504 correspond to predetermined (e.g., by a trained pathologist) locations of dopaminergic neural cells within image 1500-1540. Predetermined dopaminergic neural cells 1504 are highlighted in “blue.” Incorrectly predicted dopaminergic neural cells 1506 correspond to locations of dopaminergic neural cells within images 1500-1540 incorrectly predicted by dopaminergic neural cell segmentation and quantification model 404. Incorrectly predicted dopaminergic neural cells 1506 are highlighted in “red.” Regions of neural background tissue 1508 are highlighted in “grey” within each of images 1500-1540. As can be seen by images 1500-1540, the number of “purple” spots vastly outnumbers the number of “blue” spots and “red” spots. This strongly indicates that dopaminergic neural cell segmentation and quantification model 404 can achieve high accuracy in correctly detecting dopaminergic neural cells within an image using the techniques described herein.

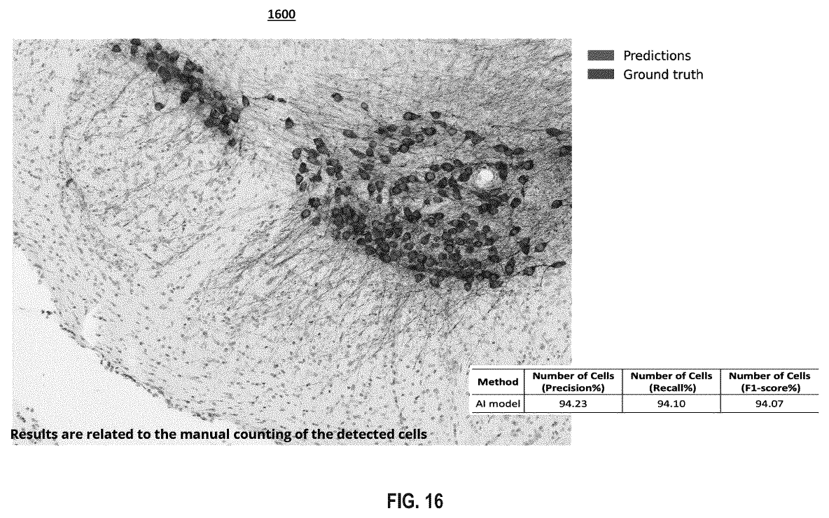

FIG. 16 is another illustrative example of the predictive power of dopaminergic neural cell segmentation and quantification model 404. For instance, as seen in image 1600 of FIG. 16, predicted dopaminergic neural cells 1602 within image 1600 correspond to dopaminergic neural cells predicted by dopaminergic neural cell segmentation and quantification model 404. Predicted dopaminergic neural cells 1602 are highlighted in “red.” Predetermined (e.g., ground truth) dopaminergic neural cells 1604 within image 1600 correspond to dopaminergic neural cells determined in advance by a trained pathologist. Predetermined dopaminergic neural cells 1604 are highlighted in “blue.” In some embodiments, dopaminergic neural cell segmentation and quantification model 404 may further be configured to determine a quantity of dopaminergic neural cells within the image. For example, dopaminergic neural cell segmentation and quantification model 404 may determine, based on the predictions, a number of dopaminergic neural cells present within an image. In some embodiments, dopaminergic neural cell segmentation and quantification model 404 may be further trained by comparing the predicted number of dopaminergic neural cells (e.g., predicted dopaminergic neural cells 1602) within an image to a precomputed number of dopaminergic neural cells within the image (e.g., ground truth dopaminergic neural cells 1604 determined manually by a trained pathologist). As seen by FIG. 16, the majority of spots (e.g., not the neural background tissue) are highlighted “purple.” This indicates that dopaminergic neural cell segmentation and quantification model 404 can achieve high accuracy when detecting dopaminergic neural cells within an image. Evidence to this effect is also illustrated in FIG. 16, which includes a table describing dopaminergic neural cell segmentation and quantification model 404 predicting the number of dopaminergic neural cells with a precision of 94.23%, a recall of 94.10%, and an F1-score of 94.07%.

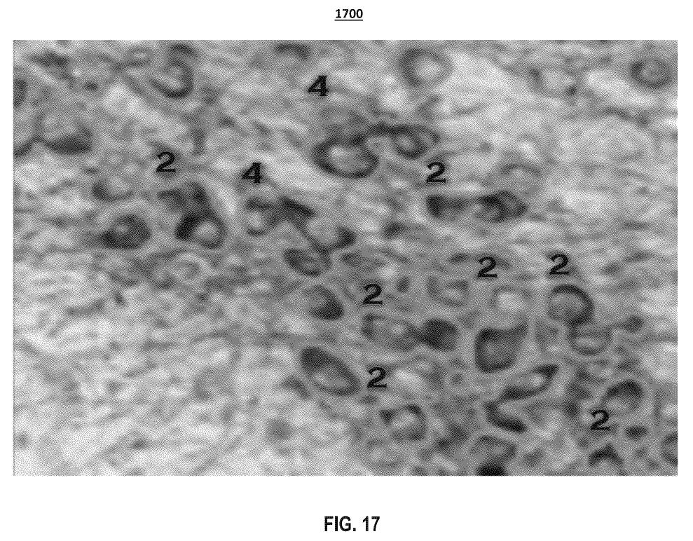

In some embodiments, neural cell segmentation and quantification subsystem 114 may be further configured to identify a plurality of clusters of pixels within the segmentation map. Each cluster may represent one or more dopaminergic neural cells within the image. As an example, with reference to FIG. 17, image 1700 depicts a zoomed-in portion of an image depicting dopaminergic neural cells. In particular, using the segmentation map (e.g., segmentation map 406 produced by dopaminergic neural cell segmentation and quantification model 404 of FIG. 4), neural cell segmentation and quantification subsystem 114 may identify clusters of dopaminergic neural cells 1702 within image 1700. In some embodiments, neural cell segmentation and quantification subsystem 114 may form an outline 1704 of a perimeter of each cluster 1702 based on the segmentation map. In FIG. 17, outlines 1704 are highlighted in “green,” however alternative colors can be used. As illustrated in FIG. 17, dopaminergic neural cell segmentation and quantification model 404 can accurately detect a location, size, and shape of dopaminergic neural cells, even those clustered together, which can improve an ability of dopaminergic neural cell segmentation and quantification model 404 to quantify dopaminergic neural cells within an image.

To count the number of dopaminergic neural cells within image 1700, neural cell segmentation and quantification subsystem 114 may be configured to determine an area of each cluster. The area may comprise a pixel area. For example, using the predicted segmentation map (e.g., segmentation map 1420 of FIG. 14), neural cell segmentation and quantification subsystem 114 may determine, for each cluster, a number of pixels occupied by that cluster. In one or more examples, neural cell segmentation and quantification subsystem 114 may determine the number of dopaminergic neural cells based on the area of each of the plurality of clusters and the plurality of clusters. In some embodiments, neural cell segmentation and quantification subsystem 114 may be configured to determine the number of dopaminergic neural cells based on the area of each of the clusters and an average size of a dopaminergic neural cell. In some embodiments, neural cell segmentation and quantification subsystem 114 may calculate the average size of a dopaminergic neural cell from training data used to train dopaminergic neural cell segmentation and quantification model 404. For example, neural cell segmentation and quantification subsystem 114 may determine, from the precomputed segmentation maps, a size/area (e.g., in pixel-space) of each detected dopaminergic neural cell. In some embodiments, a minimum size of a dopaminergic neural cell may be determined based on the size/area of each detected dopaminergic neural cell from the training data. In some embodiments, a minimum size of a dopaminergic neural cell from the training data may be selected. In some embodiments, a set of the smallest sized cells may be used to compute an approximate minimum size.

In some embodiments, neural cell segmentation and quantification subsystem 114 may be configured to determine the number of dopaminergic neural cells by filtering at least one of the clusters. In one or more examples, the clusters may be filtered based on the area of the cluster being less than a minimum size of a dopaminergic neural cell. For example, a cluster that is determined to have a size smaller than the minimum size of a dopaminergic cell, that cluster may be flagged. When neural cell segmentation and quantification subsystem 114 counts the number of dopaminergic neural cells within the image, it may ignore those clusters that have been flagged as being too small to depict a dopaminergic neural cell.

In some embodiments, neural cell segmentation and quantification subsystem 114 may be configured to determine the number of dopaminergic neural cells by identifying one or more of the plurality clusters having an area satisfying a threshold area condition. For each of the one or more of the clusters, neural cell segmentation and quantification subsystem 114 may be configured to estimate a quantity of dopaminergic neural cells represented by the cluster. In one or more examples, the number of dopaminergic neural cells may be based on the estimated quantity of dopaminergic neural cells within each cluster. For example, as seen with respect to FIG. 17, some of the clusters of dopaminergic neural cells may be larger than the average size of a dopaminergic neural cell, as determined from the training data. In some embodiments, if the area (in pixel-space) satisfies the threshold area condition, then a number of dopaminergic neural cells represented by that cluster may be determined by dividing the area by the average size of a dopaminergic neural cell. For example, if the average size of a dopaminergic neural cell is d_{avg}, then a number of cells within a cluster (satisfying the threshold area condition) may be equal to the cluster area divided by d_{avg}. As seen in FIG. 17, clusters where Area/d_{avg}˜2 may indicate that that cluster includes 2 dopaminergic neural cells. Clusters where Area/d_{avg}˜4 may indicate that that cluster includes 4 dopaminergic neural cells.

In some embodiments, the threshold area condition being satisfied may comprise the area of the cluster being greater than or equal to a threshold area. In some embodiments, the threshold area may be computed based on the average size of a dopaminergic neural cell. In some embodiments, the average size is calculated based on a size of dopaminergic neural cells identified within training data used to train the machine learning model to obtain the trained machine learning model. In some embodiments, the minimum size of the dopaminergic neural cell may be calculated based on a minimum size of the dopaminergic neural cells identified within the training data used to train the machine learning model to obtain the trained machine learning model.

Automatic cell counting can be a challenging task due to the overlapping cells which share boundaries. Thus, using the techniques described above, neural cell segmentation and quantification subsystem 114 may be capable of distinguishing overlapping cells.

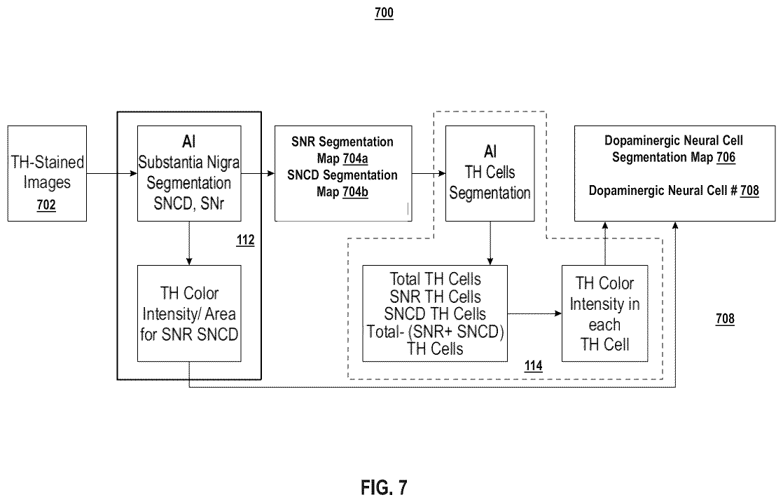

FIG. 7 illustrates an example machine learning pipeline 700 for identifying regions of SNR and SNCD within an image 702, and segmenting and quantifying dopaminergic neural cells within those regions, in accordance with various embodiments. Machine learning pipeline 700 details how SNR/SNCD segmentation subsystem 112 and neural cell segmentation and quantification subsystem 114 operate together to improve the accuracy of dopaminergic neural cell detection and quantification, which can be informative when determining treatment options.

As seen in machine learning pipeline 700, SNR/SNCD segmentation subsystem 112 may receive one or more TH-stained images 702. Images 702 may depict a section of the brain of a subject. In particular, the section of the brain depicted by images 702 may include regions of SN and, more particularly, represent where dopaminergic neural cells are to be located. In some embodiments, machine learning pipeline 700 may include TH-stained images 702 being input to SNR/SNCD segmentation subsystem 112. SNR/SNCD segmentation subsystem 112 may be configured to generate one or more segmentation maps. For example, SNR/SNCD segmentation subsystem 112 may generate an SNR segmentation map 704a and an SNCD segmentation map 704b. In one or more examples, a single SNR/SNCD segmentation map may be generated (i.e., combining the information of SNR segmentation map 704a and SNCD segmentation map 704b).

In some embodiments, SNR/SNCD segmentation subsystem 112 may also be configured to determine an intensity of the TH-stain within TH-stained image 702 and may output intensity data indicating the determined TH-stain intensity. In particular, SNR/SNCD segmentation subsystem 112 may be configured to generate intensity data by measuring an intensity of the TH-stain within TH-stained images 702. The intensity data may also include information related to an area of TH-stain image 702 encompassed by one or more regions of SNR and one or more regions of SNCD. For example, SNR/SNCD segmentation subsystem 112 may determine a number of pixels of TH-stained images 702 that have a TH-stain intensity greater than or equal to a threshold TH-stain intensity. SNR/SNCD segmentation subsystem 112 may determine an area of the regions of SNR/SNCD based on the pixels having a TH-stain intensity greater than or equal to the threshold TH-stain intensity and a size of the pixel.

In some embodiments, SNR segmentation map 704a and SNCD segmentation map 704b may be input to neural cell segmentation and quantification subsystem 114. In some embodiments, neural cell segmentation and quantification subsystem 114 may also receive TH-stained images 702. In some embodiments, neural cell segmentation and quantification subsystem 114 may be configured to generate a dopaminergic neural cell segmentation map 706 indicating a location of one or more dopaminergic neural cells identified within TH-stained images 702. In one or more examples, neural cell segmentation and quantification subsystem 114 may implement one or more machine learning models to identify dopaminergic neural cells within an input image. In particular, dopaminergic neural cell segmentation map 706 may determine a location of dopaminergic neural cells within one or more ROIs. For example, the ROIs may comprise the regions of SNR and/or the regions of SNCD. Dopaminergic neural cell segmentation map 706 may also include data for annotating TH-stained images 702 to indicate the locations and sizes of the detected dopaminergic neural cells. For example, the data may be used to display a cell outline for each detected dopaminergic neural cell.

In some embodiments, neural cell segmentation and quantification subsystem 114 may further be configured to determine a number of dopaminergic neural cells 708 within image 702. Number of dopaminergic neural cells 708 may be determined by determining the number of dopaminergic neural cells within the ROIs based on SNR segmentation map 704a and SNCD segmentation map 704b generated for each of the plurality of patches and dopaminergic neural cell segmentation map 706.

The machine learning techniques that can be used in the systems/subsystems/modules described herein may include, but are not limited to (which is not to suggest that any other list is limiting), any of the following: Ordinary Least Squares Regression (OLSR), Linear Regression, Logistic Regression, Stepwise Regression, Multivariate Adaptive Regression Splines (MARS), Locally Estimated Scatterplot Smoothing (LOESS), Instance-based Algorithms, k-Nearest Neighbor (KNN), Learning Vector Quantization (LVQ), Self-Organizing Map (SOM), Locally Weighted Learning (LWL), Regularization Algorithms, Ridge Regression, Least Absolute Shrinkage and Selection Operator (LASSO), Elastic Net, Least-Angle Regression (LARS), Decision Tree Algorithms, Classification and Regression Tree (CART), Iterative Dichotomizer 3 (ID3), C4.5 and C5.0 (different versions of a powerful approach), Chi-squared Automatic Interaction Detection (CHAID), Decision Stump, M5, Conditional Decision Trees, Naive Bayes, Gaussian Naive Bayes, Causality Networks (CN), Multinomial Naive Bayes, Averaged One-Dependence Estimators (AODE), Bayesian Belief Network (BBN), Bayesian Network (BN), k-Means, k-Medians, K-cluster, Expectation Maximization (EM), Hierarchical Clustering, Association Rule Learning Algorithms, A-priori algorithm, Eclat algorithm, Artificial Neural Network Algorithms, Perceptron, Back-Propagation, Hopfield Network, Radial Basis Function Network (RBFN), Deep Learning Algorithms, Deep Boltzmann Machine (DBM), Deep Belief Networks (DBN), Convolutional Neural Network (CNN), Deep Metric Learning, Stacked Auto-Encoders, Dimensionality Reduction Algorithms, Principal Component Analysis (PCA), Principal Component Regression (PCR), Partial Least Squares Regression (PLSR), Collaborative Filtering (CF), Latent Affinity Matching (LAM), Cerebri Value Computation (CVC), Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA), Ensemble Algorithms, Boosting, Bootstrapped Aggregation (Bagging), AdaBoost, Stacked Generalization (blending), Gradient Boosting Machines (GBM), Gradient Boosted Regression Trees (GBRT), Random Forest, Computational intelligence (evolutionary algorithms, etc.), Computer Vision (CV), Natural Language Processing (NLP), Recommender Systems, Reinforcement Learning, Graphical Models, or separable convolutions (e.g., depth-separable convolutions, spatial separable convolutions).

### Example Flowcharts

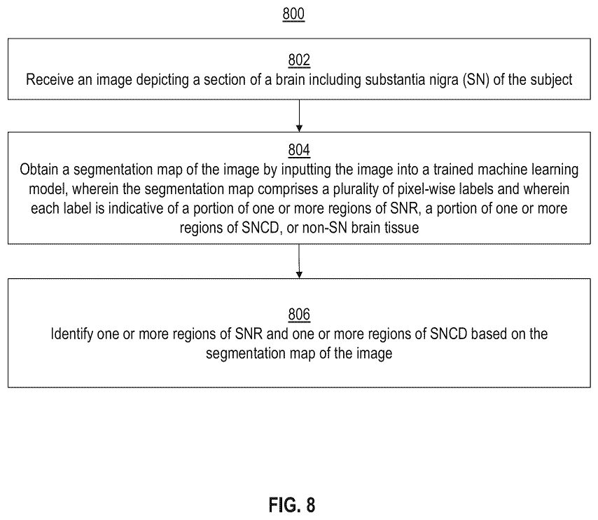

FIG. 8 illustrates a flowchart of an example method 800 for identifying regions of SNR and regions of SNCD within an image, in accordance with various embodiments. In some embodiments, method 800 may be executed by one or more computing systems. For example, method 800 may be performed by SNR/SNCD segmentation subsystem 112.

In some embodiments, method 800 may begin at step 802. At step 802, an image depicting a section of a brain including substantia nigra (SN) of a subject may be received. In some embodiments, the subject may exhibit dopaminergic neural cell loss. For example, dopaminergic neural cell loss in regions of SN of the subject has been induced externally to mimic loss of dopaminergic neurons as observed in human PD patients. In one or more examples, the section of the brain depicted by the image is stained with a stain highlighting SN. For example, the stain may be a tyrosine hydroxylase enzyme (TH). TH may be used because it is an indicator of dopaminergic neuron viability. In some embodiments, an optical density of dopaminergic neural cells within the regions of SNR and the regions of SNCD may be calculated based on an expression level of the stain within the image. For example, the stain may cause a dopaminergic neuron to turn a particular color. The intensity of that color can be quantified and used as an indication of the likelihood that a corresponding pixel of the image depicts a dopaminergic neuron. In one or more examples, the intensity of the pixel may be compared to a threshold pixel intensity. If the intensity of the pixel is greater than or equal to the threshold pixel intensity, that pixel may be classified as depicting at least a portion of a dopaminergic neuron.

At step 804, a segmentation map of the image by inputting the image into a trained machine learning model may be obtained. In one or more examples, the segmentation map comprises a plurality of pixel-wise labels. Each pixel-wise label may indicate that a corresponding pixel of the image comprises a portion of one or more regions of SNR, a portion of one or more regions of SNCD, or a portion of non-SN brain tissue. In some embodiments, the segmentation map may be generated using one or more trained machine learning models. Training the machine learning model may include, for each of a plurality of training images, extracting one or more features from the training image. In one or more examples, a feature vector representing the training image may be generated based on the one or more extracted features. One or more pixels of the training image may be classified, based on the feature vector, as representing a portion of the regions of SNR, a portion of the regions of SNCD, or a portion of non-SN brain tissue. In one or more examples, a segmentation map for the training image may be generated based on the classification of each pixel. In some embodiments, the trained machine learning model may be implemented using an encoder-decoder architecture comprising an encoder and a decoder. In one or more examples, the encoder may be configured to extract the one or more features from the training image. In one or more examples, the decoder may be configured to classify the one or more pixels of the training image. In some embodiments, the segmentation map may be generated by determining each of the pixel-wise labels based on an intensity of one or more stains applied to a biological sample of the section of the brain. The stains may be configured to highlight the regions of SNR, the regions of SNCD, and the non-SN brain tissue within the biological sample. For example, the stain may be a TH stain configured to highlight dopaminergic neural cells. In one or more examples, each pixel-wise label may indicate whether a corresponding pixel in the image depicts at least one of the regions of SNR, at least one of the regions of SNCD, or the non-SN brain tissue.

At step 806, one or more regions identify regions of substantia nigra reticulata (SNR) and one or more regions of substantia nigra compacta dorsal (SNCD) may be identified within the image based on the segmentation map of the image. In some embodiments, an annotated version of the image may be generated to indicate the identified regions of SNR and SNCD. The annotated version of the image may include a first visual indicator defining the regions of SNR within the image and a second visual indicator defining the regions of SNCD within the image.

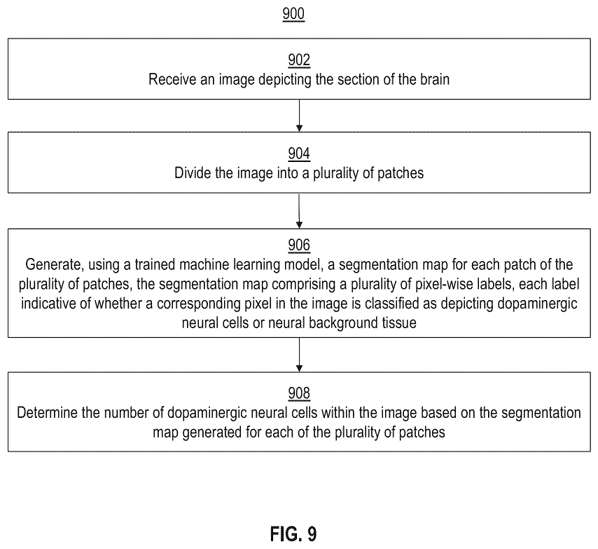

FIG. 9 illustrates a flowchart of an example method 900 for determining a number of dopaminergic neural cells within an image, in accordance with various embodiments. In some embodiments, method 900 may be executed by one or more computing systems. For example, method 900 may be performed by dopaminergic neural cell segmentation and quantification subsystem 114.

In some embodiments, method 900 may begin at step 902. At step 902, an image depicting a section of the brain of a subject may be received. In one or more examples, the subject may be diagnosed with a disease. For example, the subject may be exhibiting dopaminergic neural cell loss. For example, the subject may be diagnosed with Parkinson's disease (PD), which can cause dopaminergic neural cell loss in regions of SN. In some embodiments, a first segmentation map or segmentation maps indicating one or more ROIs within the image may be received. For example, the first segmentation map may indicate regions of SNR and/or regions of SNCD within the image.

At step 904, the image may be divided into a plurality of patches. In one or more examples, the patches are non-overlapping.

At step 906, a segmentation map for each of the patches may be generated. The segmentation map may comprise a plurality of pixel-wise labels. In one or more examples, each label may indicate whether a corresponding pixel in the image is classified as depicting dopaminergic neural cells or neural background tissue. In some embodiments, the segmentation maps may be generated using one or more trained machine learning models. In some embodiments, each of the pixel-wise labels may be determined based on an intensity of a stain applied to a biological sample of the section of the brain. In one or more examples, the stain is selected such that it highlights dopaminergic neural cells within a biological sample. In some embodiments, the pixel-wise labels may indicate whether the corresponding pixel depicts at least one SNR region and/or at least one SNCD region of the brain. For example, each pixel-wise label may indicate whether a corresponding pixel of the image depicts an SNR region or an SNCD region based on a determination that the intensity of the stain is greater than or equal to a threshold intensity.

At step 908, a number of dopaminergic neural cells within the image may be determined based on the segmentation map generated for each of the plurality of patches. In some embodiments, a plurality of clusters of pixels within the segmentation map may be identified. Each cluster may represent one or more dopaminergic neural cells within the image. In one or more examples, an area of each of the plurality of clusters may be calculated. In one or more examples, the number of dopaminergic neural cells may be based on the area of each of the plurality of clusters and the plurality of clusters. In one or more examples, the number of dopaminergic neural cells may be determined based on the area of each of the clusters and an average size of a dopaminergic neural cell. In some embodiments, the number of dopaminergic neural cells may be determined by filtering at least one of the clusters based on the area of the cluster being less than a minimum size of a dopaminergic neural cell. In some embodiments, the number of dopaminergic neural cells may be determined by identifying one or more of the plurality clusters having an area satisfying a threshold area condition. For each of the one or more of the clusters, a quantity of dopaminergic neural cells represented by the cluster may be estimated. In one or more examples, the number of dopaminergic neural cells is based on the estimated quantity of dopaminergic neural cells. In one or more examples, the area satisfying the threshold area condition may comprise the area of the cluster being greater than or equal to a threshold area. In some embodiments, the threshold area may be computed based on the average size of a dopaminergic neural cell. In some embodiments, the average size is calculated based on a size of dopaminergic neural cells identified within training data used to train the machine learning model to obtain the trained machine learning model. In some embodiments, the minimum size of the dopaminergic neural cell may be calculated based on a minimum size of the dopaminergic neural cells identified within the training data used to train the machine learning model to obtain the trained machine learning model.

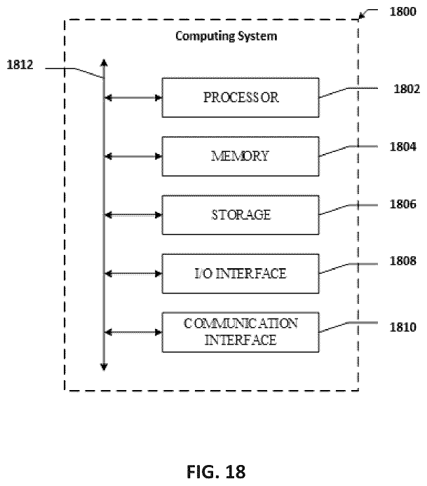

FIG. 18 illustrates an example computer system 1800. In some embodiments, one or more computer systems 1800 perform one or more steps of one or more methods described or illustrated herein. In some embodiments, one or more computer systems 1800 provide functionality described or illustrated herein. In some embodiments, software running on one or more computer systems 1800 performs one or more steps of one or more methods described or illustrated herein or provides functionality described or illustrated herein. Particular embodiments include one or more portions of one or more computer systems 1800. Herein, reference to a computer system may encompass a computing device, and vice versa, where appropriate. Moreover, reference to a computer system may encompass one or more computer systems, where appropriate.

This disclosure contemplates any suitable number of computer systems 1800. This disclosure contemplates computer system 1800 taking any suitable physical form. As example and not by way of limitation, computer system 1800 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 1800 may include one or more computer systems 1800; be unitary or distributed; span multiple locations; span multiple machines; span multiple data centers; or reside in a cloud, which may include one or more cloud components in one or more networks. Where appropriate, one or more computer systems 1800 may perform without substantial spatial or temporal limitation one or more steps of one or more methods described or illustrated herein. As an example, and not by way of limitation, one or more computer systems 1800 may perform in real time or in batch mode one or more steps of one or more methods described or illustrated herein. One or more computer systems 1800 may perform at various times or at different locations one or more steps of one or more methods described or illustrated herein, where appropriate.

In some embodiments, computer system 1800 includes a processor 1802, memory 1804, storage 1806, an input/output (I/O) interface 1808, a communication interface 1810, and a bus 1812. Although this disclosure describes and illustrates a particular computer system having a particular number of components in a particular arrangement, this disclosure contemplates any suitable computer system having any suitable number of any suitable components in any suitable arrangement.

In some embodiments, processor 1802 includes hardware for executing instructions, such as those making up a computer program. As an example, and not by way of limitation, to execute instructions, processor 1802 may retrieve (or fetch) the instructions from an internal register, an internal cache, memory 1804, or storage 1806; decode and execute them; and then write one or more results to an internal register, an internal cache, memory 1804, or storage 1806. In some embodiments, processor 1802 may include one or more internal caches for data, instructions, or addresses. This disclosure contemplates processor 1802 including any suitable number of any suitable internal caches, where appropriate. As an example, and not by way of limitation, processor 1802 may include one or more instruction caches, one or more data caches, and one or more translation lookaside buffers (TLBs). Instructions in the instruction caches may be copies of instructions in memory 1804 or storage 1806, and the instruction caches may speed up retrieval of those instructions by processor 1802. Data in the data caches may be copies of data in memory 1804 or storage 1806 for instructions executing at processor 1802 to operate on; the results of previous instructions executed at processor 1802 for access by subsequent instructions executing at processor 1802 or for writing to memory 1804 or storage 1806; or other suitable data. The data caches may speed up read or write operations by processor 1802. The TLBs may speed up virtual-address translation for processor 1802. In some embodiments, processor 1802 may include one or more internal registers for data, instructions, or addresses. This disclosure contemplates processor 1802 including any suitable number of any suitable internal registers, where appropriate. Where appropriate, processor 1802 may include one or more arithmetic logic units (ALUs); be a multi-core processor; or include one or more processors 1802. Although this disclosure describes and illustrates a particular processor, this disclosure contemplates any suitable processor.

In some embodiments, memory 1804 includes main memory for storing instructions for processor 1802 to execute or data for processor 1802 to operate on. As an example, and not by way of limitation, computer system 1800 may load instructions from storage 1806 or another source (such as, for example, another computer system 1800) to memory 1804. Processor 1802 may then load the instructions from memory 1804 to an internal register or internal cache. To execute the instructions, processor 1802 may retrieve the instructions from the internal register or internal cache and decode them. During or after execution of the instructions, processor 1802 may write one or more results (which may be intermediate or final) to the internal register or internal cache. Processor 1802 may then write one or more of those results to memory 1804. In some embodiments, processor 1802 executes only instructions in one or more internal registers or internal caches or in memory 1804 (as opposed to storage 1806 or elsewhere) and operates only on data in one or more internal registers or internal caches or in memory 1804 (as opposed to storage 1806 or elsewhere). One or more memory buses (which may each include an address bus and a data bus) may couple processor 1802 to memory 1804. Bus 1812 may include one or more memory buses, as described below. In some embodiments, one or more memory management units (MMUs) reside between processor 1802 and memory 1804 and facilitate access to memory 1804 requested by processor 1802. In some embodiments, memory 1804 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1804 may include one or more memories 3404, where appropriate. Although this disclosure describes and illustrates particular memory, this disclosure contemplates any suitable memory.

In some embodiments, storage 1806 includes mass storage for data or instructions. As an example, and not by way of limitation, storage 1806 may include a hard disk drive (HDD), a floppy disk drive, flash memory, an optical disc, a magneto-optical disc, magnetic tape, or a Universal Serial Bus (USB) drive or a combination of two or more of these. Storage 1806 may include removable or non-removable (or fixed) media, where appropriate. Storage 1806 may be internal or external to computer system 1800, where appropriate. In some embodiments, storage 1806 is non-volatile, solid-state memory. In some embodiments, storage 1806 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1806 taking any suitable physical form. Storage 1806 may include one or more storage control units facilitating communication between processor 1802 and storage 1806, where appropriate. Where appropriate, storage 1806 may include one or more storages 3406. Although this disclosure describes and illustrates particular storage, this disclosure contemplates any suitable storage.

In some embodiments, I/O interface 1808 includes hardware, software, or both, providing one or more interfaces for communication between computer system 1800 and one or more I/O devices. Computer system 1800 may include one or more of these I/O devices, where appropriate. One or more of these I/O devices may enable communication between a person and computer system 1800. As an example, and not by way of limitation, an I/O device may include a keyboard, keypad, microphone, monitor, mouse, printer, scanner, speaker, still camera, stylus, tablet, touch screen, trackball, video camera, another suitable I/O device, or a combination of two or more of these. An I/O device may include one or more sensors. This disclosure contemplates any suitable I/O devices and any suitable I/O interfaces 1808 for them. Where appropriate, I/O interface 1808 may include one or more device or software drivers enabling processor 1802 to drive one or more of these I/O devices. I/O interface 1808 may include one or more I/O interfaces 1808, where appropriate. Although this disclosure describes and illustrates a particular I/O interface, this disclosure contemplates any suitable I/O interface.

In some embodiments, communication interface 1810 includes hardware, software, or both providing one or more interfaces for communication (such as, for example, packet-based communication) between computer system 1800 and one or more other computer systems 1800 or one or more networks. As an example, and not by way of limitation, communication interface 1810 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1810 for it. As an example, and not by way of limitation, computer system 1800 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1800 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 1800 may include any suitable communication interface 1810 for any of these networks, where appropriate. Communication interface 1810 may include one or more communication interfaces 1810, where appropriate. Although this disclosure describes and illustrates a particular communication interface, this disclosure contemplates any suitable communication interface.

In some embodiments, bus 1812 includes hardware, software, or both coupling components of computer system 1800 to each other. As an example and not by way of limitation, bus 1812 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1812 may include one or more buses 4112, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

### EXAMPLE EMBODIMENTS

Embodiments disclosed herein may include:
1. A method for identifying regions of substantia nigra reticulata (SNR) and regions of substantia nigra compacta dorsal (SNCD) in images of a subject diagnosed with Parkinson's disease (PD), the method comprising: receiving an image depicting a section of a brain including substantia nigra (SN) of the subject; obtaining a segmentation map of the image by inputting the image into a trained machine learning model, wherein the segmentation map comprises a plurality of pixel-wise labels, each label indicative of whether a corresponding pixel in the image is classified as depicting a portion of one or more regions of SNR, a portion of one or more regions of SNCD, or non-SN brain tissue; and identifying one or more regions of SNR and one or more regions of SNCD based on the segmentation map of the image.
2. The method of embodiment 1, wherein the section of the brain depicted by the image is stained with a stain highlighting SN, the method further comprises: calculating an optical density of dopaminergic neural cells within the one or more regions of SNR and the one or more regions of SNCD based on an expression level of the stain within the image.
3. The method of embodiment 2, further comprising: predicting a health state of the dopaminergic neural cells within the one or more regions of SNR and the one or more regions of SNCD based on the calculated optical density.
4. The method of any one of embodiments 2-3, wherein the stain comprises a tyrosine hydroxylase enzyme (TH) stain used to determine a viability of the dopaminergic neural cells.
5. The method of any one of embodiments 1-4, wherein the trained machine learning model comprises a first trained machine learning model and the segmentation map comprises a first segmentation map, the method further comprises: generating, using a second trained machine learning model, a second segmentation map comprising a plurality of pixel-wise labels, each label indicative of whether a corresponding pixel in the image is classified as depicting dopaminergic neural cells or neural background tissue within the one or more regions of SNR and the one or more regions of SNCD.
6. The method of embodiment 5, further comprising: determining, based on the first segmentation map and the second segmentation map, a number of dopaminergic neural cells within the one or more regions of SNR and the one or more regions of SNCD or a quantity of the dopaminergic neural cells.
7. The method of any one of embodiments 1-6, wherein the trained machine learning model is trained using a plurality of training images, wherein each of the plurality of training images depicts a section of a brain including SN and includes a precomputed segmentation map corresponding to the training image.
8. The method of embodiment 7, further comprising: retrieving a plurality of images each depicting a section of a brain including SN; and performing one or more image transformation operations to each of the plurality of images to obtain the plurality of training images.
9. The method of embodiment 8, wherein the one or more image transformation operations comprise at least one of a rotation operation, a horizontal flip operation, a vertical flip operation, a random 90-degree rotation operation, a transposition operation, an elastic transformation operation, cropping, or a Gaussian noise addition operation.
10. The method of any one of embodiments 8-9, further comprising: adjusting a size of one or more of the plurality of training images such that each of the plurality of training images has a same size.
11. The method of embodiment 10, wherein the size of each of the plurality of training images is 1024× 1024 pixels.
12. The method of any one of embodiments 7-11, further comprising: training the trained machine learning model based on the plurality of training images, wherein training comprises: for each of the plurality of training images: extract one or more features from the training image; generate a feature vector representing the training image based on the one or more extracted features; classify, based on the feature vector, one or more pixels of the training image as representing a portion of the one or more regions of SNR, a portion of the one or more regions of SNCD, or a portion of non-SN brain tissue; and generate a segmentation map for the training image based on the classification.
13. The method of embodiment 12, wherein the trained machine learning model is implemented using an encoder-decoder architecture comprising an encoder and a decoder, and wherein the encoder is configured to extract the one or more features from the training image and the decoder is configured to classify the one or more pixels of the training image.
14. The method of any one of embodiments 12-13, further comprising: for each of the plurality of training images: calculating a similarity score between the segmentation map generated for the training image and the precomputed segmentation map for the training image; and adjusting one or more hyperparameters of the trained machine learning model based on the similarity score to enhance a similarity between the generated segmentation map and the precomputed segmentation map.
15. The method of any one of embodiments 1-14, further comprising: performing a first training step on the training machine learning model based on first training data comprising a plurality of non-medical images; and performing a second training step on the trained machine learning model based on second training data comprising (i) a plurality of medical images depicting sections of the brain including SN and (ii) a precomputed segmentation map for each of the plurality of medical images.
16. The method of embodiment 15, wherein the precomputed segmentation map for each of the plurality of medical images comprises a plurality of pixel-wise labels, each label being indicative of a portion of one or more regions of SNR, a portion of one or more regions of SNCD, or non-SN brain tissue, and wherein the second training step is performed after the first training step.
17. The method of any one of embodiments 1-16, further comprising: generated an annotated version of the image comprising a first visual indicator defining the one or more regions of SNR within the image and a second visual indicator defining the one or more regions of SNCD within the image.
18. The method of any one of embodiments 1-17, wherein generating the segmentation map comprises: determining each of the plurality of pixel-wise labels based on an intensity of one or more stains applied to a biological sample of the section of the brain, wherein the one or more stains are configured to highlight the one or more regions of SNR, the one or more regions of SNCD, and the non-SN brain tissue within the biological sample, wherein the pixel-wise label indicates that a corresponding pixel in the image depicts at least one of the one or more regions of SNR region, at least one of the one or more regions of SNCD, or the non-SN brain tissue.
19. A non-transitory computer-readable medium storing computer program instruction that, when executed by one or more processors, effectuate the method of any one of embodiments 1-18.
20. A system for identifying regions of substantia nigra reticulata (SNR) and regions of substantia nigra compacta dorsal (SNCD) in images of a subject diagnosed with Parkinson's disease (PD), the system comprising: one or more processors programmed to perform the method of any one of embodiments 1-18.
21. A method for determining a number of dopaminergic neural cells within an images depicting a section of a brain of a subject diagnosed with Parkinson's disease (PD), the method comprising: receiving an image depicting the section of the brain; dividing the image into a plurality of patches; generating, using a trained machine learning model, a segmentation map for each patch of the plurality of patches, the segmentation map comprising a plurality of pixel-wise labels, each label indicative of whether a corresponding pixel in the image is classified as depicting dopaminergic neural cells or neural background tissue; and determining the number of dopaminergic neural cells within the image based on the segmentation map generated for each of the plurality of patches.
22. The method of embodiment 21, wherein the segmentation map comprises a first segmentation map and the trained machine learning model comprises a first trained machine learning model, the method further comprises: receiving a second segmentation map indicating one or more regions of interest (ROIs) within the image from a second trained machine learning model, wherein determining the number of dopaminergic neural cells within the image comprises: determining the number of dopaminergic neural cells within the one or more ROIs based on the first segmentation map generated for each of the plurality of patches and the second segmentation map.
23. The method of embodiment 22, wherein the second segmentation map is computed prior to the first segmentation map being generated, and wherein the one or more ROIs indicate at least one substantia reticular (SNR) region of the brain or at least one substantia nigra compacta dorsal (SNCD) region of the brain.
24. The method of any one of embodiments 22-23, further comprising: determining each of the plurality of pixel-wise labels based on an intensity of a stain applied to a biological sample of the section of the brain, the stain being configured to highlight dopaminergic neural cells within a biological sample, wherein the pixel-wise label indicates that the corresponding pixel depicts at least one SNR region of the brain based on the intensity of the stain being greater than or equal to a threshold intensity or the pixel-wise label indicates that the corresponding pixel depicts at least one SNCD region of the brain based on the intensity of the stain being less than the threshold intensity.
25. The method of embodiment 24, further comprising: determining a health state of the dopaminergic neural cells based on the intensity of the stain expressed by each pixel of the image classified as depicting a dopaminergic neural cell.
26. The method of any one of embodiments 21-25, further comprising: training the trained machine learning model to recognize dopaminergic neural cells within an input image, wherein training comprises: performing a first self-supervised learning (SSL) step to an encoder based on first training data comprising a first plurality of non-medical images to obtain a first trained encoder; and performing a second SSL step to the first trained encoder based on second training data comprising (i) a second plurality of images each depicting a section of a brain comprising at least one substantia reticular (SNR) region or at least one substantia nigra compacta dorsal (SNCD) region and (ii) first predetermined segmentation maps comprising a plurality of pixel-wise labels, each label indicative of whether a corresponding pixel in a corresponding image of the second plurality of images is classified as depicting a dopaminergic neural cell or neural background tissue to obtain a second trained encoder.
27. The method of embodiment 26, wherein training further comprises: performing a supervised learning step to the second trained encoder based on third training data comprising (i) a third plurality of images each depicting a section of a brain comprising at least one substantia reticular (SNR) region or at least one substantia nigra compacta dorsal (SNCD) region and (ii) second predetermined segmentation maps comprising a plurality of pixel-wise labels, each label indicative of whether a corresponding pixel in a corresponding image of the third plurality of images is classified as depicting a dopaminergic neural cell or neural background tissue.
28. The method of any one of embodiments 26-27, wherein performing the first SSL step comprises: for each of the first plurality of non-medical images: dividing the image into a plurality of patches; for each of the plurality of patches: generating a first augmented view of the patch and a second augmented view of the patch; generating, using a first instance of the encoder comprising a first plurality of hyperparameters, a first embedding representing the first augmented view; generating, using a second instance of the encoder comprising a second plurality of hyperparameters, a second embedding representing the second augmented view; calculating a difference between the first embedding and the second embedding; and adjusting one or more of the first plurality of hyperparameters based on the calculated difference.
29. The method of any one of embodiments 26-28, wherein performing the second SSL step comprises: for each of the second plurality of images: dividing the image into a plurality of patches; for each of the plurality of patches: generating a first augmented view of the patch and a second augmented view of the patch; generating, using a first instance of the first trained encoder comprising a first plurality of hyperparameters, a first embedding representing the first augmented view; generating, using a second instance of the first trained encoder comprising a second plurality of hyperparameters, a second embedding representing the second augmented view; calculating a difference between the first embedding and the second embedding; and adjusting one or more of the first plurality of hyperparameters based on the calculated difference.
30. The method of embodiment 28 or 29, wherein calculating the difference comprises: computing a cross-correlation matrix based on the first embedding and the second embedding, and wherein the one or more of the first plurality of hyperparameters are adjusted to minimize off-diagonal elements of the cross-correlation matrix and normalize diagonal elements of the cross-correlation matrix.
31. The method of embodiment 28 or 29, wherein generating the first augmented view and the second augmented view comprise applying one or more image transformation operations to the patch, the one or more image transformation operations comprising at least one of: a flip operation, a rotation operation, a RGB shift operation, a blurring operation, a Gaussian noise augmentation operation, or a cropping operation.
32. The method of any one of embodiments 21-31, wherein the plurality of patches are non-overlapping.
33. The method of any one of embodiments 21-32, further comprising: identifying a plurality of clusters of pixels within the segmentation map, wherein each cluster represents one or more dopaminergic neural cells; and calculating an area of each of the plurality of clusters, wherein the number of dopaminergic neural cells is based on the area of each of the plurality of clusters and the plurality of clusters.
34. The method of embodiment 33, wherein determining the number of dopaminergic neural cells comprises: determining the number of dopaminergic neural cells based on the area of each of the plurality of clusters and an average size of a dopaminergic neural cell.
35. The method of embodiment 34, wherein determining the number of dopaminergic neural cells comprises: filtering at least one of the plurality of clusters based on the area of the at least one cluster being less than a minimum size of a dopaminergic neural cell.
36. The method of any one of embodiments 34-35, wherein determining the number of dopaminergic neural cells comprises: identifying one or more of the plurality of clusters having an area satisfying a threshold area condition; for each of the one or more of the plurality of clusters: estimating a quantity of dopaminergic neural cells represented by the cluster, wherein the number of dopaminergic neural cells is based on the estimated quantity of dopaminergic neural cells.
37. The method of embodiment 36, wherein the area satisfying the threshold area condition comprises: the area of the cluster being greater than or equal to a threshold area, the threshold area being computed based on the average size of a dopaminergic neural cell.
38. The method of any one of embodiments 35-37, wherein: the average size is calculated based on a size of dopaminergic neural cells identified within training data used to train the trained machine learning model; and the minimum size of the dopaminergic neural cell is calculated based on a minimum size of the dopaminergic neural cells identified within the training data used to train the trained machine learning model.
39. A non-transitory computer-readable medium storing computer program instruction that, when executed by one or more processors, effectuate the method of any one of embodiments 21-38.
40. A system for determining a number of dopaminergic neural cells within an image depicting a section of a brain of a subject diagnosed with Parkinson's disease (PD), the system comprising: one or more processors programmed to perform the method of any one of embodiments 21-38.

1. A method for identifying regions of substantia nigra reticulata (SNR) and regions of substantia nigra compacta dorsal (SNCD) in images of a subject diagnosed with Parkinson's disease (PD), the method comprising:
receiving an image depicting a section of a brain including substantia nigra (SN) of the subject;
obtaining a segmentation map of the image by inputting the image into a trained machine learning model, wherein the segmentation map comprises a plurality of pixel-wise labels, each label indicative of whether a corresponding pixel in the image is classified as depicting a portion of one or more regions of SNR, a portion of one or more regions of SNCD, or non-SN brain tissue; and
identifying one or more regions of SNR and one or more regions of SNCD based on the segmentation map of the image.

2. The method of claim 1, wherein the section of the brain depicted by the image is stained with a stain highlighting SN, the method further comprises:
calculating an optical density of dopaminergic neural cells within the one or more regions of SNR and the one or more regions of SNCD based on an expression level of the stain within the image.

3. The method of claim 2, further comprising:
predicting a health state of the dopaminergic neural cells within the one or more regions of SNR and the one or more regions of SNCD based on the calculated optical density.

4. The method of claim 2, wherein the stain comprises a tyrosine hydroxylase enzyme (TH) stain used to determine a viability of the dopaminergic neural cells.

5. The method of claim 1, wherein the trained machine learning model comprises a first trained machine learning model and the segmentation map comprises a first segmentation map, the method further comprises:
generating, using a second trained machine learning model, a second segmentation map comprising a plurality of pixel-wise labels, each label indicative of whether a corresponding pixel in the image is classified as depicting dopaminergic neural cells or neural background tissue within the one or more regions of SNR and the one or more regions of SNCD.

6. The method of claim 5, further comprising:
determining, based on the first segmentation map and the second segmentation map, a number of dopaminergic neural cells within the one or more regions of SNR and the one or more regions of SNCD or a quantity of the dopaminergic neural cells.

7. The method of claim 1, wherein the trained machine learning model is trained using a plurality of training images, wherein each of the plurality of training images depicts a section of a brain including SN and includes a precomputed segmentation map corresponding to the training image.

8. The method of claim 7, further comprising:
retrieving a plurality of images each depicting a section of a brain including SN; and
performing one or more image transformation operations to each of the plurality of images to obtain the plurality of training images.

9. The method of claim 8, wherein the one or more image transformation operations comprise at least one of a rotation operation, a horizontal flip operation, a vertical flip operation, a random 90-degree rotation operation, a transposition operation, an elastic transformation operation, cropping, or a Gaussian noise addition operation.

10. The method of claim 8, further comprising:
adjusting a size of one or more of the plurality of training images such that each of the plurality of training images has a same size.

11. The method of claim 10, wherein the size of each of the plurality of training images is 1024× 1024 pixels.

12. The method of claim 7, further comprising:
training the trained machine learning model based on the plurality of training images, wherein training comprises:
for each of the plurality of training images:
extract one or more features from the training image;
generate a feature vector representing the training image based on the one or more extracted features;
classify, based on the feature vector, one or more pixels of the training image as representing a portion of the one or more regions of SNR, a portion of the one or more regions of SNCD, or a portion of non-SN brain tissue; and
generate a segmentation map for the training image based on the classification.

13. The method of claim 12, wherein the trained machine learning model is implemented using an encoder-decoder architecture comprising an encoder and a decoder, and wherein the encoder is configured to extract the one or more features from the training image and the decoder is configured to classify the one or more pixels of the training image.

14. The method of claim 12, further comprising:
for each of the plurality of training images:
calculating a similarity score between the segmentation map generated for the training image and the precomputed segmentation map for the training image; and
adjusting one or more hyperparameters of the trained machine learning model based on the similarity score to enhance a similarity between the generated segmentation map and the precomputed segmentation map.

15. The method of claim 1, further comprising:
performing a first training step on the training machine learning model based on first training data comprising a plurality of non-medical images; and
performing a second training step on the trained machine learning model based on second training data comprising (i) a plurality of medical images depicting sections of the brain including SN and (ii) a precomputed segmentation map for each of the plurality of medical images.

16. The method of claim 15, wherein the precomputed segmentation map for each of the plurality of medical images comprises a plurality of pixel-wise labels, each label being indicative of a portion of one or more regions of SNR, a portion of one or more regions of SNCD, or non-SN brain tissue, and wherein the second training step is performed after the first training step.

17. The method of claim 1, further comprising:
generated an annotated version of the image comprising a first visual indicator defining the one or more regions of SNR within the image and a second visual indicator defining the one or more regions of SNCD within the image.

18. The method of claim 1, wherein generating the segmentation map comprises:
determining each of the plurality of pixel-wise labels based on an intensity of one or more stains applied to a biological sample of the section of the brain, wherein the one or more stains are configured to highlight the one or more regions of SNR, the one or more regions of SNCD, and the non-SN brain tissue within the biological sample, wherein the pixel-wise label indicates that a corresponding pixel in the image depicts at least one of the one or more regions of SNR region, at least one of the one or more regions of SNCD, or the non-SN brain tissue.

19. A non-transitory computer-readable medium storing computer program instruction that, when executed by one or more processors, cause the one or more processors to:
receive an image depicting a section of a brain including substantia nigra (SN) of the subject;
obtain a segmentation map of the image by inputting the image into a trained machine learning model, wherein the segmentation map comprises a plurality of pixel-wise labels, each label indicative of whether a corresponding pixel in the image is classified as depicting a portion of one or more regions of SNR, a portion of one or more regions of SNCD, or non-SN brain tissue; and
identify one or more regions of SNR and one or more regions of SNCD based on the segmentation map of the image.

20. A system for identifying regions of substantia nigra reticulata (SNR) and regions of substantia nigra compacta dorsal (SNCD) in images of a subject diagnosed with Parkinson's disease (PD), the system comprising one or more processors programmed to:
receive an image depicting a section of a brain including substantia nigra (SN) of the subject;
obtain a segmentation map of the image by inputting the image into a trained machine learning model, wherein the segmentation map comprises a plurality of pixel-wise labels, each label indicative of whether a corresponding pixel in the image is classified as depicting a portion of one or more regions of SNR, a portion of one or more regions of SNCD, or non-SN brain tissue; and
identify one or more regions of SNR and one or more regions of SNCD based on the segmentation map of the image.

21.-40. (canceled)
